(19) United States
(12) Patent Application Publication
(10) Pub. No.: US 2026/0290624 A1
(43) Pub. Date: Sep. 24, 2026

(54) MATCHING TELEMETRY DATA AND VIDEO DATA OF A MEDICAL PROCEDURE RECEIVED BY A COLLABORATIVE MEDICAL PLATFORM TO A MEDICAL PRACTITIONER

(71) Applicant: Cilag GmbH International, Zug (CH)

(72) Inventors: Shan Gowri Jegatheeswaran, Santa Clara, CA (US); Raymond Samuel Fryrear, II, Cincinnati, OH (US); Fiona Middlemiss Haig, York (GB); Alexandre Hennen, New York, NY (US); Vikram Mohan, San Francisco, CA (US); Christine Purcell, New York, NY (US)

(21) Appl. No.: 19/689,863

(22) Filed: May 27, 2026

Related U.S. Application Data
(63) Continuation of application No. PCT/IB2024/062186, filed on Dec. 4, 2024.
(60) Provisional application No. 63/605,879, filed on Dec. 4, 2023.
(60) Provisional application No. 63/641,754, filed on May 2, 2024.
(60) Provisional application No. 63/661,015, filed on Jun. 17, 2024.
(60) Provisional application No. 63/661,858, filed on Jun. 19, 2024.
(60) Provisional application No. 63/717,950, filed on Nov. 8, 2024.
(60) Provisional application No. 63/718,000, filed on Nov. 8, 2024.
(60) Provisional application No. 63/719,015, filed on Nov. 11, 2024.

(51) Int. Cl. G16H 80/00; G16H 50/20

(52) U.S. Cl. CPC G16H 80/00; G16H 50/20

(57) ABSTRACT

A collaborative medical platform facilitates remote collaboration relating to medical procedures during preprocedural, intraprocedural, and postprocedural stages of a medical case. The collaborative medical platform receives telemetry data or video data from a location that does not identify a medical practitioner or a medical procedure. Using characteristics of medical practitioners maintained by the collaborative medical platform and the telemetry data or video data, the collaborative medical platform selects a medical practitioner connected to the telemetry data or video data. The collaborative medical platform stores a connection between the selected medical practitioner and the telemetry data or video data. Subsequently, the collaborative medical platform may request supplemental information about the telemetry data or video data from the selected medical practitioner.

### CROSS-REFERENCE TO RELATED APPLICATIONS

This application is a continuation of PCT Application No. PCT/IB2024/062186 filed on Dec. 4, 2025, which claims the benefit of the following applications: U.S. Provisional Patent Application No. 63/605,879 filed on Dec. 4, 2023, U.S. Provisional Patent Application No. 63/641,754 filed on May 2, 2024, U.S. Provisional Patent Application No. 63/661,015 filed on Jun. 17, 2024, U.S. Provisional Patent Application No. 63/661,858 filed on Jun. 19, 2024, U.S. Provisional Patent Application No. 63/717,950 filed on Nov. 8, 2024, U.S. Provisional Patent Application No. 63/718,000 filed on Nov. 8, 2024, and U.S. Provisional Patent Application No. 63/719,015 filed on Nov. 11, 2024. The contents of each of the foregoing applications are each incorporated by reference herein.

### BACKGROUND

### Technical Field

The described embodiments relate to a system and method for matching telemetry data and video data of a medical procedure received by a collaborative medical platform to a medical practitioner.

### Description of the Related Art

During many medical procedures, sensors capture telemetry data describing operation or settings of one or more pieces of medical equipment used during a medical procedure. Additionally, one or more cameras or image capture devices capture video data describing performance of the procedures. Capturing telemetry data and video data allows subsequent evaluation of performance of the medical procedure. Such evaluation allows identification of changes to one or more techniques for performing the medical procedure or identification of techniques performed during the medical procedure to distribute to medical practitioners who performed the medical procedure or other medical practitioners who perform the type of medical procedure.

To aid evaluation of telemetry data or video data, a location where a medical procedure is performed may provide captured telemetry data or video data to an external system allowing for more in-depth analysis of the telemetry data or video data. Using such an external system may also simplify subsequent leverage of the telemetry data or video data by other medical practitioners accessing the external system. However, data privacy restrictions often limit information that locations, such as medical facilities, can distribute outside of a medical facility where a medical procedure was performed. For example, many medical facilities are prevented from providing external systems with information capable of uniquely identifying a patient on whom a medical procedure is performed. To comply with such data privacy restrictions, a location cannot include information identifying a medical procedure or identifying one or more medical practitioners associated with the medical procedure to an external system with telemetry data or video data captured during the medical procedure. Similarly, compliance with data privacy restrictions prevents a location where a medical procedure was performed from providing an external system with access to scheduling information for medical procedures performed at the location.

Without receiving or accessing information identifying participants in a medical procedure from a location (e.g., a medical facility), an external system is unable to correlate received telemetry data or video data for a medical procedure with a medical practitioner performing the medical procedure, or otherwise associated with the medical procedure. To associate telemetry data or video data captured during a medical procedure with a medical practitioner, conventional external systems use manual review of received telemetry data or video data to identify a medical procedure and a medical practitioner associated with the telemetry data or video data. As locations, such as medical facilities, often perform a large number of medical procedures each day, an amount of telemetry data or video data, as well as information about medical practitioners and medical procedures, to be reviewed for correlating telemetry data or video data with medical practitioners is impractical and unwieldly for manual review. Hence, compliance with data privacy restrictions by locations often prevents external systems from providing feedback to medical practitioners based on telemetry data or video data, as an external system is often unable to ascertain one or more medical practitioners associated with the telemetry data or video data.

### BRIEF DESCRIPTION OF THE DRAWINGS

FIG. 1 is an example embodiment of a computing environment for an electronically-assisted medical procedure.

FIG. 2 is a block diagram of an example architecture for a collaborative medical platform.

FIG. 3A shows a first view of an example practitioner dashboard associated with a collaborative medical platform.

FIG. 3B shows a second view of an example practitioner dashboard associated with a collaborative medical platform.

FIG. 4 shows an example practitioner dashboard displaying an educational content item to a medical practitioner associated with a collaborative medical platform.

FIG. 5 is an example embodiment of a case sharing interface associated with sharing a medical case in the collaborative medical platform.

FIG. 6 is an example embodiment of a case dashboard associated with a set of cases in a collaborative medical platform.

FIG. 7 is an example telepresence interface associated with a collaborative medical platform.

FIG. 8 is another example of a telepresence interface associated with a collaborative medical platform.

FIG. 9 is an example analytics dashboard associated with a collaborative medical platform.

FIG. 10 is an example video interface associated with a collaborative medical platform.

FIG. 11 is an example prompt for a medical practitioner to confirm a connection to telemetry data or video data.

FIGS. 12A-2C are example interfaces requesting supplemental information for telemetry data or video data connected to a medical practitioner.

FIG. 13 is a flowchart of an example embodiment of a process for a collaborative medical platform selecting a medical practitioner connected to received telemetry data or video data.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A collaborative medical platform facilitates exchange of data between remote medical practitioners in relation to medical cases during preprocedural, intraprocedural, and postprocedural stages. The collaborative medical platform receives telemetry data or video data captured during performance of a medical procedure from a medical facility. The telemetry data describes movement or operation of one or more pieces of medical equipment during the medical procedure, and the video data captures actions of one or more medical practitioners during the medical procedure. However, to comply with one or more data privacy restrictions, the collaborative medical platform does not receive data identifying a medical practitioner performing the medical or identifying a patient on whom the medical procedure was performed.

To associate the telemetry data or video data with a medical practitioner, the collaborative medical platform leverages information about medical practitioners maintained by the collaborative medical platform, as well as information received from client devices associated with medical practitioners to select a medical practitioner for received telemetry data or video data. For example, the collaborative medical platform trains a practitioner prediction model to receive telemetry data or video data along with characteristics of a medical practitioner and to generate a probability of the medical practitioner being associated with the telemetry data or the video data. Based on the probabilities generated for at least a set of medical practitioners, the collaborative medical platform selects a medical practitioner and transmits a prompt to a client device of the selected medical practitioner that includes descriptive information from the video data or the telemetry data and a request for the medical practitioner to confirm a connection between the selected medical practitioner and the video data or the telemetry data. In response to receiving a confirmation of the connection to the video data or the telemetry data, the collaborative medical platform may also receive supplemental information about the medical procedure corresponding to the telemetry data or the video data. The collaborative medical platform stores the connection between the telemetry data or the video data, as well as supplemental information from the selected medical practitioner, and the selected medical practitioner. This allows the collaborative medical platform to provide the selected medical practitioner with metrics or other information from the collaborative medical platform's analysis of the video data or the telemetry data.

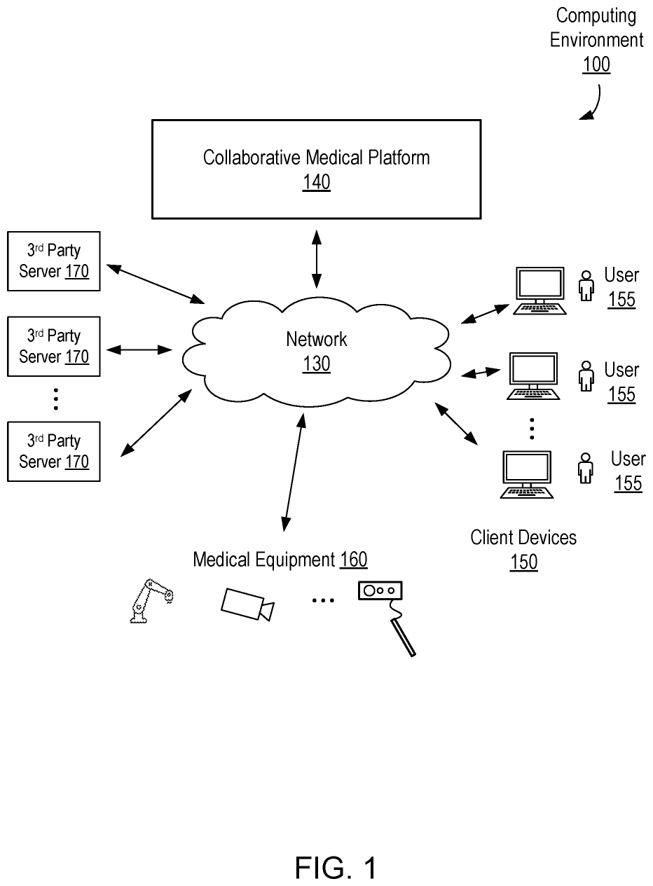

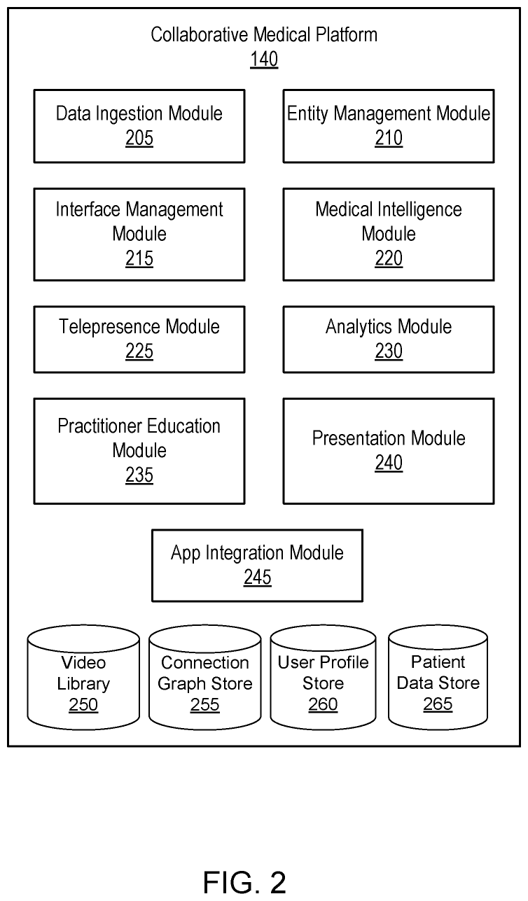

FIG. 1 illustrates an example embodiment of a computing environment 100 for a collaborative medical platform 140. The collaborative medical platform 140 may include one or more servers that are coupled by a network 130 to client devices 150 associated with users 155 of the collaborative medical platform 140, medical equipment 160, and various third-party servers 170. The collaborative medical platform 140 facilitates collaborative exchange of data between medical practitioners, patients, administrators, or other users 155 via the client devices 150 in support of preprocedural, intraprocedural, and postprocedural stages of medical cases. The collaborative medical platform 140 may furthermore facilitate access to telemetry data from medical equipment 160 (including, for example, real-time video, images, biometric sensing data, equipment control and/or status signals, etc.) that may be utilized in conjunction with performing medical procedures and managing patient cases. Furthermore, the collaborative medical platform 140 may facilitate access to various third-party servers 170 that provide external services such as, for example, electronic healthcare records (EHR) services, medical telepresence services, operating room scheduling, data analytics services, etc.

To further support the preprocedural stage of a medical case, the collaborative medical platform 140 may select one or more reference content items for presentation to a medical practitioner before performing a medical procedure. In various embodiments, the collaborative medical platform 140 maintains a store or a library of reference content items from which reference content items for the medical practitioner are selected. Alternatively or additionally, one or more third-party servers 170 maintain reference content items, and the collaborative medical platform 140 selects one or more reference content items from a third-party server 170. Reference content items for a medical practitioner may be retrieved from a combination of one or more third-party servers 170 and the collaborative medical platform 140. The collaborative medical platform 140 leverages data in a user profile of a medical practitioner to select one or more reference content items for the medical practitioner.

In support of an intraprocedural stage of a medical case, the collaborative medical platform 140 may facilitate presentation of various information to support the procedure such as preprocedural images, models, patient data, equipment information, or other data. The collaborative medical platform 140 may furthermore facilitate a telepresence session that enables one or more remote contributors to access video, images, 3D models, equipment telemetry data, or other data streams capturing during an ongoing medical procedure. The collaborative medical platform 140 may furthermore enable remote practitioners to provide annotations or other commentary related to real-time video, images, or three-dimensional models associated with a procedure. The collaborative medical platform 140 tracks and stores all data from the procedure (including video, medical equipment telemetry, and collaborative commentary) in association with the case identifier to enable subsequent access.

During the intraprocedural stage, the collaborative medical platform 140 may present educational content about a medical procedure being performed to one or more medical practitioners performing the medical procedure. Educational content describes performance of the medical procedure, such as information about techniques to use, movement of medical instruments or medical equipment, settings for medical equipment, or other information. The collaborative medical platform 140 compares telemetry data or video data of a medical procedure during the intraprocedural stage to baseline criteria associated with educational content and selects educational content associated with baseline criteria from which the telemetry data or video data deviates. Educational content may include instructions that, when executed by a piece of medical equipment 160, modify one or more settings of the piece of medical equipment based on the educational content, simplifying adjustment of operation of the piece of medical equipment 160.

In support of a postprocedural stage of a medical procedure, the collaborative medical platform 140 enables medical practitioners connected with a case to collaboratively monitor data associated with a patient's recovery. For example, the collaborative medical platform 140 may provide interfaces for viewing health records associated with the patient's recovery and facilitate collaborative exchange between medical practitioners through a case-specific content feed. The collaborative medical platform 140 may furthermore perform various analytics relating to performed medical procedures based on aggregations of data. The analytics may be useful to support patient recovery, to improve future procedures, and to track the performance of medical practitioners.

Educational content relevant to a medical procedure may be selected and presented to a medical practitioner who performed the medical procedure during the postprocedural stage by the collaborative medical platform 140. For example, metrics or analytics determined for the medical procedure by the collaborative medical platform 140 are compared to baseline criteria for various educational content. In various embodiments, the collaborative medical platform 140 selects educational content associated with baseline criteria from which a metric deviates and presents the selected educational content to the medical practitioner. For example, the collaborative medical platform 140 includes information identifying selected educational content in one or more interfaces generated for presentation to the medical practitioner, simplifying access to instructional information relative to the medical procedure.

The collaborative medical platform 140 may intelligently utilize data collected during preprocedural, intraprocedural, and/or postprocedural stages of a case during a different stage of the same case or other cases. For example, annotations of images or 3D models, practitioner comments from a content feed, or other information obtained during a preprocedural stage may be made available in the intraprocedural stage to aid the performing practitioner through the procedure. Analytical data relating to postprocedural data may be utilized to generate recommendations for future procedures, such as educational content, in order to improve efficiencies and/or outcomes.

The collaborative medical platform 140 may also facilitate functions such as managing clinical trials, facilitating education training and performance tracking, facilitating broadcasts of medical-related presentations, and facilitating procedure scheduling. Beneficially, the collaborative medical platform 140 stores complete records of medical cases (including video and telemetry from procedures) in a centralized and standardized platform that naturally allows for collaboration in an online environment, where practitioners may interact from disparate remote locations. The collaborative medical platform 140 may maintain data in a manner that adheres to data privacy and compliance obligations of medical practitioners and organizations.

The collaborative medical platform 140 may furthermore employ various machine learning techniques to infer recommendations, insights, or other artificially generated contributions based on the data collected into the collaborative medical platform 140. For example, the collaborative medical platform 140 may generate a recommendation for a medical practitioner to review educational content relevant to a medical practitioner based on data captured by the collaborative medical platform 140 during performance of a medical procedure. For example, the collaborative medical platform 140 selects educational content for a medical practitioner based on telemetry data captured during a medical procedure performed by the medical practitioner. As another example, the collaborative medical platform selects educational content for a medical practitioner based on video data captured during a medical procedure performed by the medical practitioner. Educational content selected by the collaborative medical platform may be video, audio, text, or other data describing performance of a medical procedure. Additionally or alternatively, educational content configuration instructions or configuration data for one or more pieces of medical equipment 160.

The collaborative medical platform 140 may generate and present other recommendations to a medical practitioner based on stored information for the medical practitioner. For example, the collaborative medical platform 140 generates a recommendation for the medical practitioner based on a type of procedure scheduled to be performed by the medical practitioner; in various embodiments, the recommendation comprises case records associated with one or more historical cases captured in the collaborative medical platform 140 relating to prior performances of the type of procedure on a similarly situated patient. If granted appropriate permissions, the practitioner may then review an entire case record through the collaborative medical platform 140 including preprocedural information, videos or other data from the procedure itself, and postprocedural outcome data. In another example, the collaborative medical platform 140 may intelligently generate a recommendation to invite a particular medical practitioner to collaborate on a case based on that practitioner having relevant expertise, experience, and/or availability. An invitation may then be generated to the medical practitioner to enable access and collaboration on the case during at least one of the preprocedural, intraprocedural, and postprocedural stages. Furthermore, the collaborative medical platform 140 may intelligently identify and present patient risk factors relevant to procedure performance, planning, and postprocedural care. The collaborative medical platform 140 may also intelligently recommend educational content for training medical practitioners based on their individual tracked performance and various comparative analytics.

The collaborative medical platform 140 may be implemented using on-site computing or storage systems, cloud computing or storage systems, or a combination thereof and may be implemented utilizing local or cloud-based servers, which may include physical or virtual machines, or a combination thereof. Cloud-based servers may include private cloud systems, public cloud systems, hybrid public/private cloud systems, or a combination thereof. Accordingly, the collaborative medical platform 140 may be local, remote, and/or distributed relative to the medical environments where procedures are performed and relative to the client devices 150 providing user access. Furthermore, different portions of the collaborative medical platform 140 may execute on different remote servers and various system elements of the collaborative medical platform 140 may be communicatively coupled over a network 130.

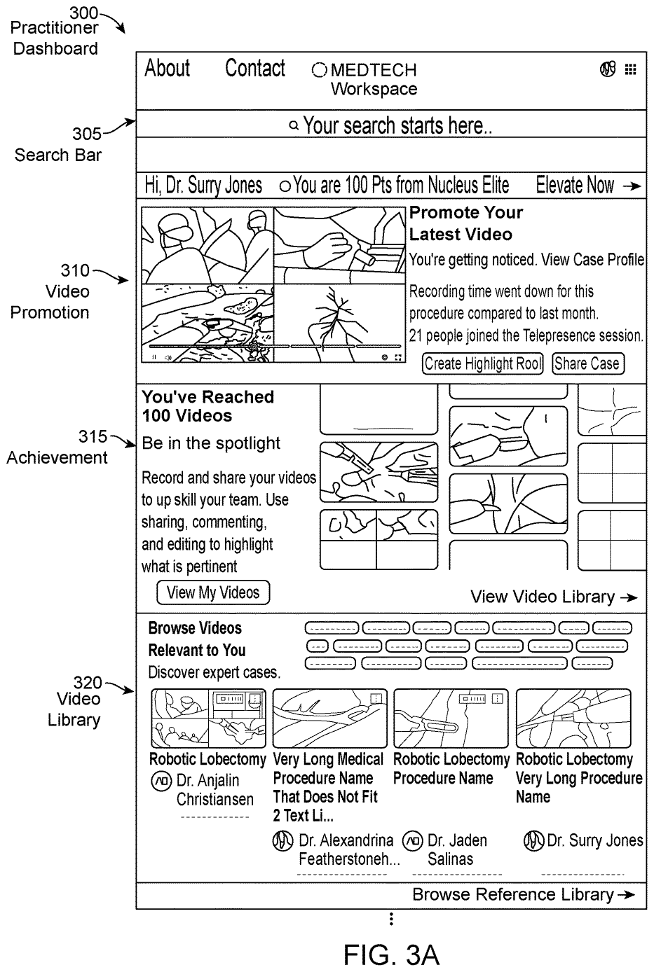

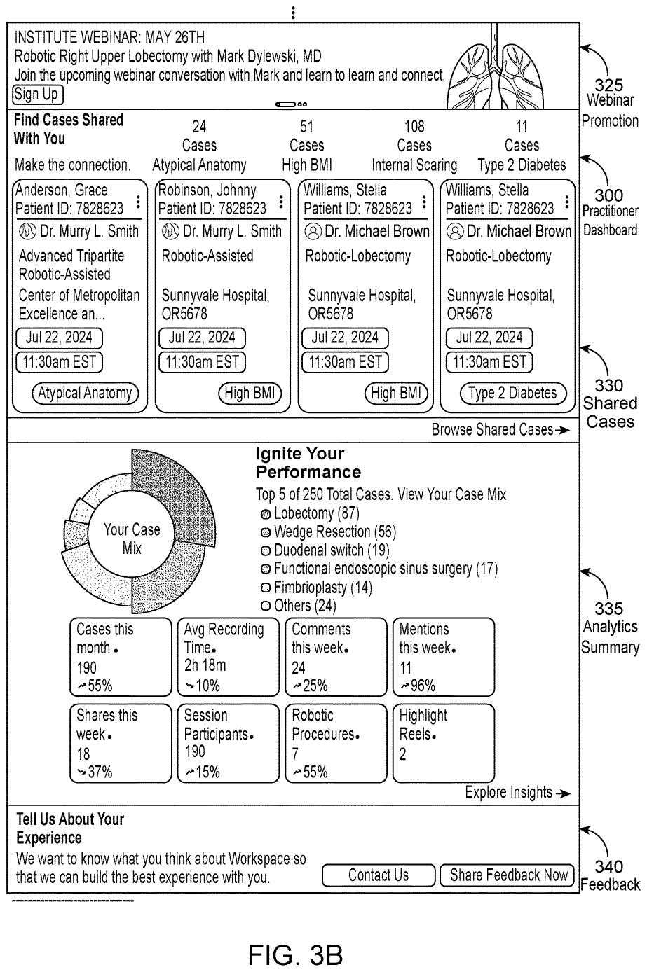

The client devices 150 may include, for example, a mobile phone, a tablet, a laptop or desktop computer, other computing device, or application executing thereon for accessing the collaborative medical platform 140 via the network 130. The client devices 150 may enable access to various user interfaces (which may comprise web-based interfaces accessed via a browser or application interfaces accessed via an application) for viewing and/or editing information associated with the collaborative medical platform 140. The client devices 150 may include conventional computer hardware such as a display, input device (e.g., touch screen), memory, a processor, and a non-transitory computer-readable storage medium that stores instructions for execution by the processor in order to carry out functions described herein. Examples of user interfaces are described in further detail below with respect to FIGS. 3-12C.

The third-party servers 170 may facilitate diverse services utilized by the collaborative medical platform 140. For example, the third-party servers 170 may include various EHR systems for managing patient records, robotic control platforms for controlling surgical robots or other medical equipment, telepresence servers for facilitating telepresence services, patient scheduling systems, hospital information systems (HIS), or other servers. As another example, one or more third-party servers 170 include educational content about various medical procedures, such as articles about various medical procedures, audio data related to medical procedures, video data related to medical procedures, settings or configuration details for medical equipment 160 used in medical procedures, or other descriptive information about medical procedures. The third-party servers 170 may be implemented using various on-site computing or storage systems, cloud computing or storage systems such as private cloud systems, public cloud systems, hybrid public/private cloud systems, or a combination thereof.

The medical equipment 160 may include various sensors such as cameras or other imaging equipment, biometric monitors, or other sensing devices that collect data associated with a medical procedure being performed. Sensor data may include physiological or biological signals (such as pulse rate, blood pressure, body temperature, etc.), video, electrical signals representative of a state of a medical instruction, or other information. Cameras or image sensors may include still image cameras, video cameras, 3-dimensional (3D) imaging devices, or a combination thereof. The cameras can include stationary cameras in a medical environment (e.g., operating room) or may include cameras integrated into medical instruments such as endoscopic cameras. Imaging systems may include computed tomography (CT) imaging systems, medical resonance imaging (MRI) systems, X-ray systems, or other imaging equipment. The medical equipment may furthermore include a robotic device that facilitates robotically-assisted medical procedures. The robotic device may include, for example, a robotic arm or other computer-controlled mechanical device that performs or assists with a medical procedure. The robotic device may be pre-programmed to perform a certain set of steps or tasks, and/or may be manually controlled by an operator. Telemetry data associated with a robotic device may include force data, positional data, or other sensor data, control signals, fault conditions, or other data relating to operation of the robotic device during a procedure. The medical equipment data may be streamed to the collaborative medical platform 140 in real-time or may be stored on a third-party server 170 and later uploaded to the collaborative medical platform 140.

The network 130 comprises communication pathways for communication between the collaborative medical platform 140, the medical equipment 160, the client devices 150, and the third-party servers 170. The network 130 may include one or more local area networks and/or one or more wide area networks (including the Internet). The network 130 may also include one or more direct wired or wireless connections (e.g., Ethernet, WiFi, cellular protocols, WiFi direct, Bluetooth, Universal Serial Bus (USB), or other communication link).

FIG. 2 is a block diagram showing an example architecture of an embodiment of the collaborative medical platform 140. In the embodiment of FIG. 2, the collaborative medical platform 140 includes a data ingestion module 205, an entity management module 210, an interface management module 215, a medical intelligence module 220, a telepresence module 225, an analytics module 230, a practitioner education module 235, a presentation module 240, an application integration module 245, a video library 250, a connection graph store 255, a user profile store 260, and a patient data store 265. In other embodiments, the collaborative medical platform 140 includes different or additional functional blocks than those shown in FIG. 2. Further, in some embodiments, a single functional block provides the functionality of multiple functional blocks shown in FIG. 2.

While in one embodiment, the illustrated functional blocks may execute entirely within the collaborative medical platform 140, alternative embodiments may include various modules or discrete functions of modules being executed by one or more third-party servers 170. Here, the collaborative medical platform 140 may interact with a third-party server 170 via an application programming interface (API) to enable the collaborative medical platform 140 to request and utilize services provided by the third-party servers 170 to facilitate any of the functions described herein. For example, in an embodiment, electronic health records may be provided by a third-party server 170. Here, the collaborative medical platform 140 may query the third-party server 170 for relevant data but does not necessarily locally store complete patient records. Furthermore, third-party servers 170 may facilitate services such as telepresence sessions, presentation creation, access to video resources, three-dimensional model generation, or other aspects of the functions of the collaborative medical platform 140 described herein.

The data ingestion module 205 ingests various medical data used by the collaborative medical platform 140. The data ingestion module 205 may be electronically coupled to one or more external servers, databases, or other data sources that supply the medical data. The medical data may include, for example, profile data for patients (e.g., demographic information, health history, etc.), medical professionals (e.g., expertise, experience, etc.), or facilities, information about medical conditions, procedures, and medications, information about robotic systems, imaging systems, intervention tools, or other medical equipment, information about post-procedural outcomes, or other medical information discussed herein.

The data ingestion module 205 may aggregate data from various input data sources. For example, the data ingestion module 205 may obtain medical data from conventional electronic health records (EHR) systems. Here, the data ingestion module 205 may perform various pre-processing to normalize data to a standardized format used by the collaborative medical platform 140. For example, medical records may be organized in a database structure that includes values (strings, numerical values, binary values, or other data types) assigned to each of a set of predefined information fields.

The data ingestion module 205 may furthermore interface with one or more imaging systems to ingest preprocedural, intraprocedural, or postprocedural images, video, or three-dimensional models associated with patients. For example, the data ingestion module 205 may obtain and store X-ray images, magnetic resonance imaging (MRI) images, computed tomography (CT) scan images, visible light images, near infrared fluorescent (NIRF) images, or other medical images, video, or three-dimensional models derived from them. Image data may furthermore include image or video data from one or more cameras present in a medical environment where a medical procedure is being performed, such as one or more overhead cameras and/or one or more endoscopic cameras. Imaging data may include associated metadata such as telemetry data from one or more medical instruments used to perform the medical procedure, annotations or commentary associated with the video received from one or more medical practitioners associated with the medical procedure, segmentation data associated with dividing a video into segments relating to different steps of a procedure, or other information relating to image or video data.

To simplify subsequent retrieval and review of video of a medical procedure along with associated metadata, the data ingestion module 205 may perform various preprocessing and indexing of the content and associated metadata. For example, the data ingestion module 205 indexes video of a medical procedure with associated metadata to correlate different metadata with different portions of the video, synchronize videos associated with the same medical procedure, or perform various encoding or reformatting of video data. Videos may furthermore be automatically segmented and indexed into video segments corresponding to different steps of a procedure.

The data ingestion module 205 may furthermore integrate with various robotic platforms or other medical equipment to obtain telemetry data associated with procedures. For example, the data ingestion module 205 may obtain various sensor data from sensors utilized during medical procedures, identifying information associated with medical equipment, control data associated with control a robotic platform or other medical equipment, or other data generated from medical equipment in associated with performed medical procedures.

The data ingestion module 205 may furthermore provide interfaces accessible via the client device 150 for ingesting data input directly into the collaborative medical platform 140. For example, the data ingestion module 205 may present various forms or freeform entry elements to enable entry of medical information relevant to operation.

In an embodiment, the data ingestion module 205 may manage data in a manner consistent with various compliance and privacy policies. For example, the data ingestion module 205 may enable removal or redaction of portions of received data to preserve privacy of a patient when the data is used for purposes in which patient identification is not necessary.

The entity management module 210 manages presentation of entity pages associated with different entities affiliated with the collaborative medical platform 140 and manages connections between entities. Entities may include, for example, users 155 (which may medical practitioners, patients, administrators, etc.), medical cases associated with procedures, facilities, medical equipment 160, files or media content, events (e.g., conferences), presentations, training modules, or other data objects. Entity pages may comprise web pages accessible via a web browser of the client device 150 or may comprise pages of a desktop or mobile application installed on a client device 150.

Each entity page for an entity may enable viewing of information associated with the entity and/or interactions with the entity. For example, each user 155 of the collaborative medical platform may have a dedicated page that provides information about the user 155 such as identifying information, role (e.g., surgeon, nurse, executive, administrator, patient, etc.) profile information (e.g., biography, credentials, etc.), assigned cases, procedure histories, connections to other users or cases, scheduling information, or other user-specific data. An entity page for a patient (whether or not the patient is a user 155 of the collaborative medical platform 140) may include patient profile information, health history, planned procedures, risk factors, or the medical information associated with the patient. An entity page for a medical case may include information about a patient associated with the case, descriptive information about a medical procedure (such as a type of medical procedure) associated with the case, a medical environment where the medical procedure is to be performed, other descriptive information about the medical procedure, a status of the procedure (e.g., preprocedural stage, intraprocedural stage, or postprocedural stage), or other information relevant to a medical case. Pages may furthermore include various interactive elements (e.g., content feeds) that enable users to share and interact with data associated with that entity as will be further described below.

The entity management module 210 also organizes pages and associated data received into the collaborative medical platform 140 into a connection graph (stored to the connection graph store 255) that captures relationships between different entities and associated data. Some connections may be configured as default connections, while other connections may be created based on specific actions from users 155. For example, users 155 may be connected by default to other users 155 (with at least viewing permissions) within the same organization. Alternatively, connections may be generated only when a user 155 expressly invites another user 155 to connect and the receiving user 155 accepts the connection request. Connections between medical practitioners and medical cases may similarly be created by default or in response to invitations to create a connection. For example, a default connection may be created between an entry for a planned medical procedure and a medical practitioner assigned responsibility for the procedure. Alternatively, all medical practitioners within an organization or within a relevant department may become connected to a planned procedure as a default. In other scenarios, a user may share a medical case with one or more other medical practitioners to generate a connection request that invites the other medical practitioners to collaborate with on the medical case. Accepting the connection request may then create a connection between the invited practitioner and the medical case. Supplemental connections may also automatically be generated (e.g., between the owner of the procedure and the invited contributor). Connections may furthermore be created between users 155 and individual videos, files, presentations, or other data objects. For example, a user 155 that creates or owns a video may share the video with one or more other users 155 to grant access rights to the video.

Connections between entities may be of diverse types and may be governed by different permissions. Generally, pages may be accessed only by users having appropriate access permissions. Different permission levels may dictate distinct levels of access for different pages. For example, depending on user-specific permissions for a particular page, the user may be permitted or blocked from accessing the data, editing the data, commenting or annotating the data, deleting the data, or performing other modifications. In an embodiment, a page may have a page owner with the highest level of access permissions. Generally, a medical practitioner may be the page owner for their own profile page and for procedures for which they have primary responsibility. Pages associated with facilities, medical equipment, or other entities may variably be owned by an assigned medical practitioner. Non-owners may have distinct levels of access to pages depending on the configured permissions. Permissions may be granted by the page owner or by another user that has appropriate permissions to assign or relinquish permissions to other users.

Based on different connections available to different users 155, the collaborative medical platform 140 enables a personalized experience for each user 155. For example, upon logging into the collaborative medical platform 140, a user 155 may be presented with personalized interfaces that relate to their connections to other users 155, medical cases, videos, presentations, or other content hosted by the collaborative medical platform 140.

An interface management module 215 manages content associated with various interfaces hosted by the collaborative medical platform 140 and accessible via the client devices 150. As described above, the interface management module 215 may manage pages associated with the various entities managed by the collaborative medical platform 140 including users 155 (which may include medical practitioners, patients, administrators, etc.), medical cases associated with procedures, facilities, medical equipment 160, files or media content, events (e.g., conferences), presentations, training modules, or other data objects. Access to different pages by a specific user 155 may be dependent on that user's connections and permissions configured in the connection graph store 255. Furthermore, patient data may be pseudonymized for viewing by certain other users (dependent on the type of connection and/or permission) such that the patient data cannot be attributed to a specific individual.

A medical case page associated with a medical case may include information organized into preprocedural, intraprocedural, and postprocedural stages. At the preprocedural stage, a medical case page may include information about the patient, the procedure being performed, and the medical practitioner performing the procedure. The interface management module 215 may furthermore provide access to various analytical information (e.g., generated by the analytics module 230 described below) such as risk factors for the patient, experience/expertise of the medical practitioner, outcomes for the type of procedure being planned, or other data. At the intraprocedural stage, the medical case page may provide access to a telepresence session to enable remote collaborators to remotely collaborate with respect to an ongoing procedure. At a postprocedural stage, the medical case page may include information about the patient treatment plan, risk factors, follow up visits, or other postprocedural information.

Some entity pages in the collaborative medical platform 140 may include content feeds to facilitate collaboration between users 155. Content feeds may include various content (e.g., posts) such as text-based commentary, images, video, three-dimensional models, or other multimedia content relating to a medical case. Content may be directly posted to a page associated with a medical case or a post may comprise links to content stored by the collaborative medical platform 140 or on an external server. Posts may be grouped into conversations that hierarchically track the relationships between posts. For example, posts may be made as original posts (which start a new conversation) or as replies to existing posts (which become part of the conversation).

In an example use, a user 155 may invite one or more other users 155 to collaborate on a medical case and thereby gain access to a case page for the medical case. A content feed on the page enables the collaborating users 155 to post to the case page in association with the medical case. The content feed may therefore enable discussion about the procedure to be performed discussion of risk, best practices, or other information that may be useful to the practitioner performing the procedure. Furthermore, the contributing users 155 may post videos or three-dimensional models (or links to content) relating to historical procedures for similarly situated patients. Additionally, contributing users 155 could share links to entity pages associated with past procedures that may be of relevance, to enable a performing medical practitioner to view historical content feeds associated with those procedures. Patient data may optionally be pseudonymized when shared with other users (dependent on the type of connection and/or permission) such that the patient data cannot be attributed to a specific individual.

Content feeds may furthermore be utilized in relation to an ongoing procedure during a real-time telepresence session as discussed in further detail below. Here, a content feed may be presented as a real-time chat window that enables contributors to comment during a procedure, share video, images, or other media, provide links to relevant resources, or otherwise contribute content during the course of procedure.

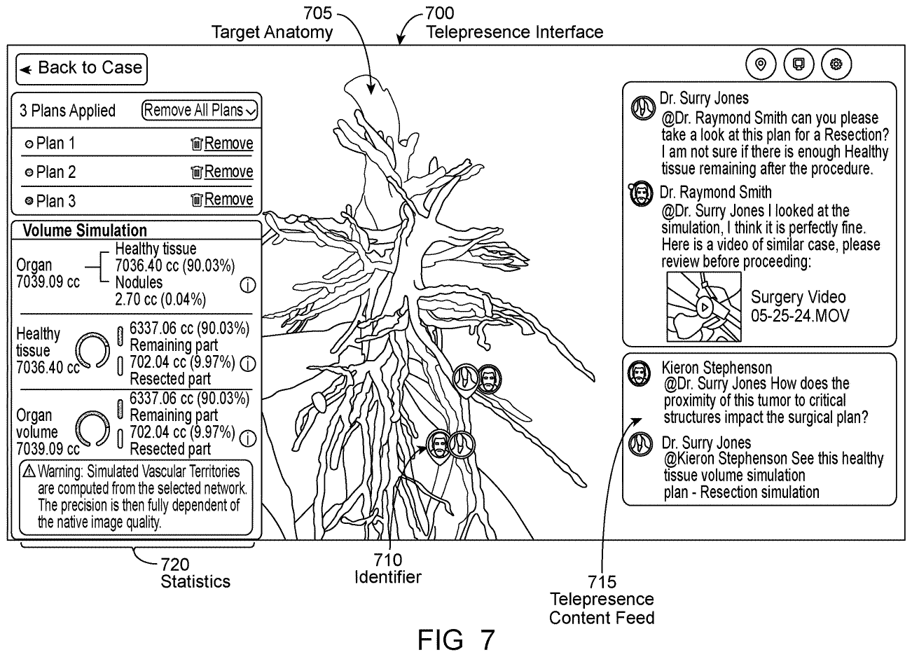

In a postprocedural stage, a content feed may be utilized by contributors to discuss postprocedural treatments, patient recovery, risk management, or other information relevant to patient recovery. Examples of content feeds are provided in FIG. 7 which are described in further detail below.

The medical intelligence module 220 generates medical intelligence data that may be automatically added to content feeds or otherwise made available in the context of the collaborative medical platform 140. For example, the medical intelligence module 220 may automatically contribute posts to a content feed for a medical case that an artificial intelligence agent infers is relevant. Artificially generated posts may mimic posts provided by human contributors and may include text-based commentary, multimedia, links, etc. Medical intelligence data may be generated during a preprocedural stage, during a procedure, or during a postprocedural stage.

In an example implementation, the medical intelligence module 220 may include one or more machine-learned models trained to generate content that the models infer to be relevant to a particular medical case or more generally relevant to a user 155. In one implementation, the machine learned model generates an embedding for a medical case based on descriptive information about a medical procedure, characteristics of the patient on whom the medical procedure is to be performed, characteristics of medical practitioner performing the procedure, posts in the content feed, or other information available in the collaborative medical platform 140. The medical intelligence module 220 determines measures of similarity (e.g., cosine similarity, dot product) between the embedding for the medical case and embeddings for other content available in the collaborative medical platform 140 and that can be included in automated posts. The medical intelligence module 220 may then generate posts and/or select content for posts based on similarities of the embeddings. The medical intelligence module 220 may furthermore employ various Large Language Models (LLMs) to analyze text-based content associated with a medical case and artificially generate relevant natural language content for the content feed. Machine learning models may furthermore include one or more neural networks (such as convolutional neural network (CNN), artificial neural network (ANN), residual neural network (ResNet), or recurrent neural network (RNN)), regression-based models, generative models, or other type of machine-learned model capable of achieving the functions described herein.

In an example use case, the medical intelligence module 220 may identify one or more historical medical cases that are similar to a current medical case and automatically generate a link to a case page for the related case. A medical practitioner may then view videos, models, or other recorded data associated with the related medical case to help the practitioner prepare for a procedure. In other examples, the medical intelligence module 220 may automatically respond to a question posed by a user in the content feed. For example, the medical intelligence module 220 may operate like a chatbot that intelligently responds to text-based queries. In further embodiments, the medical intelligence module 220 may generate a recommendation to invite a specific medical practitioner to collaborate on a medical case based on relevant expertise and experience. A user may then select to invite the recommended collaborator to collaborate on the medical case based on the artificially generated recommendation.

The telepresence module 225 facilitates a telepresence session during a procedure. The telepresence session may be joined by one or more collaborators that have been invited to collaborate on the medical case and enable the other users 155 to remotely access video, telemetry data from one or more medical instruments, or other real-time data captured during a medical procedure. As described above, a content feed may also be displayed in association with the telepresence session to enable contributors to comment or share multimedia or links relevant to the procedure.

The telepresence module 225 may furthermore enable contributors to provide real-time annotations on images, video, three-dimensional models, or other visual content of anatomy relevant to an ongoing procedure. For example, a contributor may mark locations in the visual content in association with provided comments. The telepresence module 225 may furthermore enable contributors to add overlaid drawings, highlighting, or other visual indicators during an ongoing telepresence session.

In an embodiment, the telepresence module 225 may enable remote contributors to take control of medical equipment 160. For example, a remote contributor may access a control interface that provides control elements for controlling a position or orientation of a camera, controlling a robotic arm, setting a configuration of a sensing device, or performing other control functions of medical equipment.

Upon completing a procedure, the telepresence module 225 may store the recorded video, telemetry data, content feed, annotations, and other captured data in association with the procedure. This information may be later accessed by users 155 of the collaborative medical platform 140 (with appropriate permissions) and/or may be utilized by the medical intelligence module 220 to further train machine learning models and/or generate inferences.

The analytics module 230 facilitates generation of various statistics, metrics, or other analytics associated with information stored in the collaborative medical platform 140. Analytics may generally be created based on a set of filtering parameters that yield some subset of data records for aggregating, and a combining function that specifies how the filtered data should be combined. The filtering parameters may filter medical procedure data based on data fields such as patient data, medical practitioner data, facility, procedure type, medical equipment used, etc. The combining function may comprise, for example, an averaging function, a median function, a histogram function, or other function. A specific analytics function may result in a single output value or a series of values over one or more dimensions. Series outputs may be visually represented in a table, chart, graph, or other visual output.

For example, the analytics module 230 may generate metrics describing an average length of time for a specific medical practitioner or a group of medical practitioners to complete a medical procedure. Average times for various procedures performed by the same medical practitioner or group of practitioners may be presented together with similar metrics for other medical practitioners for comparison purposes. In another example, the analytics module 230 may generate metrics describing a number of times a medical practitioner has historically performed a specific type of medical procedure. Such counts could be further aggregated to indicate percentages that reflect how many times a medical practitioner has performed each different type of medical procedure out of a total number of procedures performed.

In further embodiments, the analytics module 230 may generate analytics based on interactions of the medical practitioner in the collaborative medical platform 140. For example, statistics can be derived based on counts of posts, comments, or other content contributed by a medical practitioner to the collaborative medical platform 140. Such analytics may be expressed in terms of counts of interactions, frequency of interactions, or other aggregations. These analytics could furthermore be separately aggregated based on whether interactions relate to preprocedural, intra-procedural, or post-procedural phases of procedures.

In an embodiment, the analytics module 230 may generate analytics based on specific filtering and/or combining functions specified by a user 155 of the collaborative medical platform 140. Additionally, the analytics module 230 may include various preset analytics that may be generated without necessarily receiving specific user inputs. Furthermore, in some embodiments, the medical intelligence module 220 may automatically generate analytics that it infers will be relevant to a specific user 155.

In some embodiments, the analytics module 230 may generate analytics based on any aspects of the collective case data including preprocedural data, telepresence sessions data (including recorded video, telemetry data, in-session content feed data, etc.), and postprocedural data. Analysis associated with telepresence session data may include performing various video processing, content recognition, or other advanced image processing techniques to extract useful information from videos. Furthermore, the analytics module 230 may leverage various medical intelligence data generated from the medical intelligence module 220 to generate analytics.

The analytics module 230 also receives telemetry data from sensors captured during performance of a medical procedure. The sensors may be included in one or more pieces of medical equipment 160 used during the medical procedure or may be external to the pieces of medical equipment 160. Additionally or alternatively, the analytics module 230 receives video data captured from one or more cameras (or image capture devices) of the medical procedure being performed. Telemetry data describes how a piece of medical equipment 160 was used during a medical procedure. For example, a piece of medical equipment 160 is a robot, and the telemetry data includes configuration information of the robot or data captured by one or more sensors describing movement or operation of the robot during the medical procedure (e.g., changes in position of the robot at different times, a force applied by the robot at different times, a rate at which the robot changed position, inputs received by the robot at different times, etc.). Different sensors may capture different types of telemetry data during a medical procedure, or different sensors may capture telemetry data from different pieces of medical equipment 160.

One or more cameras, or other image capture devices, included in a location where a medical procedure is performed capture video data of the medical procedure being performed. For example, cameras are positioned at different locations within an operating room where one or more medical procedures are performed and capture different portions of the operating room. The video data includes one or more medical practitioners performing the medical procedure, and may include portions of one or more pieces of medical equipment 160 used during the medical procedure, one or more medical instruments used during the medical procedure, a portion of a patient on whom the medical procedure is being performed, or other information about the medical procedure. Multiple cameras may capture different video data of the medical procedure, with different cameras capturing different portions of the medical procedure.

In various embodiments, the telemetry data or video data includes metadata identifying a location from which the telemetry data or video data was captured, as well as times when the telemetry data or video data was captured. For example, telemetry data or video data includes a name of the location (e.g., the name of the medical facility) where the medical procedure was performed. Further, the analytics module 230 may generate metadata associated with telemetry data or video data through analysis of the telemetry data or video data. For example, the analytics module 230 applies one or more models to the telemetry data or video data to extract features of the telemetry data or video data that are stored as metadata associated with the telemetry data or video data. Example features extracted from the telemetry data or video data include: a location associated with the telemetry data or video data, a type of medical procedure during which the telemetry data or video data was captured, one or more pieces of medical equipment 160 associated with the telemetry data or video data.

However, metadata included in telemetry data or video data often does not include information identifying one or more medical practitioners who performed the medical procedure or descriptive information about the medical procedure. Many medical facilities are subject to data privacy restrictions on information transmitted to systems external to the medical facilities. Data privacy restrictions prevent (or may complicate) a medical facility from transmitting data including information capable of uniquely identifying a patent on whom a medical procedure was performed to systems (e.g., servers) in one or more locations external to the medical facility, so such data privacy restrictions prevent inclusion of metadata identifying the medical practitioner or identifying the medical procedure in telemetry data or video data transmitted to the collaborative medical platform 140. Excluding metadata identifying a medical practitioner or a medical procedure from telemetry data or video data transmitted to the collaborative medical platform 140 complies with one or more data privacy restrictions applicable to a medical facility by excluding information that directly and uniquely identify a patient based on the medical practitioner and the medical procedure. While omitting metadata identifying the medical practitioner or the medical procedure complies with data privacy restrictions imposed on the medical facility, such omission prevents the analytics module 230 from directly having access to information expressly identifying the medical practitioner performing (or associated with) the medical procedure during which the telemetry data or video data was captured.

Without directly receiving information identifying the medical procedure or identifying a medical practitioner associated with the medical procedure, the analytics module 230 may operate to indirectly infer connections of received telemetry data or video data to a medical practitioner. Inferring this connection enables the medical practitioner associated with the medical procedure during which the telemetry data or video data was captured to receive metrics or analytics about the medical procedure determined by the analytics module 230. This medical practitioner can then obtain information from the analytics module 230 for refining subsequent performance of the type of medical procedure during which the telemetry data or video data was captured, based on these inferred connections.

To select a medical practitioner to connect with telemetry data or video data, the analytics module 230 leverages the telemetry data or the video data as well as data from one or more of: the user profile store 260, the video library 250, and the connection graph store 255. Example characteristics of a medical practitioner from the medical practitioner's user profile include: one or more locations (e.g., medical facilities) associated with the medical practitioner, one or more types of medical procedures associated with the medical practitioner, information describing performance of one or more medical procedures by the medical practitioner, as well as other information describing types of medical procedures associated with the medical practitioner. Video data in the video library 250 connected to the user profile of a medical practitioner via the connection graph store 255 is one or more characteristics of the medical practitioner in some embodiments.

Additionally, a user profile stored for a medical practitioner includes one or more procedure cards associated with the medical practitioner. Procedure cards associated with the medical practitioner are characteristics of the medical practitioner that the analytics module 230 may use to select a medical practitioner to connect with telemetry data or video data. Each procedure card in a user profile of a medical practitioner is associated with a type of medical procedure. Different procedure cards may be associated with different types of medical procedures. In some embodiments, the user profile store 260 includes a set of procedure cards associated with a type of medical procedure for the medical practitioner, with each procedure card of the set associated with a step occurring during performance of the type of medical procedure.

A procedure card includes preferences, techniques or methods for performing a type of medical procedure associated with the medical practitioner. For example, a procedure card associated with a type of medical procedure specifies one or more specific medical instruments the medical practitioner uses for a step of the type of medical procedure. The procedure card may also specify positioning of different medical instruments or pieces of medical equipment 160 within a location where the medical practitioner performs the medical procedure for a step of the type of medical procedure, so a procedure card specifies placement of medical instruments or pieces of medical equipment 160 for the medical practitioner when performing different steps in the type of medical procedure. Further, the user profile for a medical practitioner includes the set of procedure cards for a type of medical procedure in an order that corresponds to order in which the medical practitioner performs different steps of the type of medical procedure; hence, procedure cards with higher positions in the set correspond to steps performed at earlier times in the type of medical procedure.

Further, based on telemetry data or video data previously received and associated with the medical practitioner, the analytics module 230 determines usage patterns for different pieces of medical equipment 160 or for different medical instruments and may determine patterns of movement of a medical practitioner from the telemetry data or video data. For example, the analytics module 230 applies one or more machine learning models to video data or to telemetry data to identify pieces of medical equipment 160 or medical instruments included in the video data or the telemetry data, to identify patterns of movement of a piece of medical equipment 160 or of a medical instrument from the video data or the telemetry data, to identify patterns of values for one or more settings of a piece of medical equipment 160, to identify patterns of movement of a medical practitioner or of a piece of medical equipment 160 from the telemetry data or video data, or to identify other descriptive information in the telemetry data. The analytics module 230 may store patterns detected from telemetry data or from video data associated with a medical practitioner with at least a threshold frequency in association with the medical practitioner and with a type of medical procedure associated with the telemetry data or video data. Associating certain patterns of movement with a medical practitioner and with a type of medical procedure in a user profile of a medical practitioner allows identification of patterns or movement or usage typical to performance of the type of medical procedure for the medical practitioner.

Subsequently, the analytics module 230 may apply one or more classification models to received telemetry data or video data that determine a type of medical procedure during which the telemetry data or video data was captured based on patterns of movement of the medical practitioner or of pieces of medical equipment. A classification model may leverage patterns of movement of a medical practitioner or of one or more pieces of medical equipment from stored video data or telemetry data associated with one or more medical practitioners to determine a type of medical procedure during which the telemetry data or video data was captured. For example, the video library 250 associates a type of medical procedure with different stored video data or telemetry data. A classification model determines a type of medical procedure from which the telemetry data or video data was captured based on measures of similarity to stored video data associated with different types of medical procedures, which accounts for different patterns of movement of pieces of medical equipment 160 or of a medical practitioner when performing different types of medical procedures. For example, the classification model is a nearest neighbor model that generates an embedding for telemetry data or video data and embeddings for stored video data associated with different types of medical procedures. Such a classification model determines a measure of similarity between the embedding for the telemetry data or video data and each embedding for stored video data. The classification model determines a type of medical procedure for the telemetry data or video data as a type of medical procedure associated with stored video data having an embedding with a maximum measure of similarity to the embedding for the telemetry data or video data. In other embodiments, the classification model determines distances between the embedding for the telemetry data or video data and each embedding for stored video data and determines a type of medical procedure for the telemetry data or video data as a type of medical procedure associated with video data having an embedding with a minimum distance to the embedding of the telemetry data or video data.

Applying a classification model to the telemetry data or video data and stored video data allows patterns of movement of pieces of medical equipment 160 or patterns of movement of a medical practitioner stored in the video library 250 to be leveraged to identify a type of medical procedure from which telemetry data or video data was captured. The determined type of medical procedure may be stored as metadata in association with the telemetry data or video data and used as input to the practitioner probability model. The determined type of medical procedure during which the telemetry data or video data was captured may be used when selecting a medical practitioner connected to the telemetry data or video data, allowing types of medical procedures associated with medical practitioners to be compared to the type of medical procedure during which the telemetry data or video data was captured.

Additionally, a user profile associated with a medical practitioner includes a log of times when the medical practitioner accessed the collaborative medical platform 140 via a client device 150. For example, the log identifies a date and a time when the medical practitioner accessed the collaborative medical platform 140 via a client device 150, and may include an identifier of a client device 150 through which the medical practitioner accessed the collaborative medical platform 140. The log may also include an identifier of a location (e.g., a medical facility) from which a client device 150 of the medical practitioner accessed the collaborative medical platform 140. Further, the user profile associated with the medical practitioner may include an indication whether the medical practitioner is currently accessing the collaborative medical platform 140 via a client device 150.

In various embodiments, the analytics module 230 trains a practitioner prediction model to generate a probability of a medical practitioner being connected to telemetry data or video data based on the telemetry data or video data and characteristics of the medical practitioner from a user profile of the medical practitioner. Example characteristics of a medical practitioner include: one or more medical facilities associated with the medical practitioner, one or more types of medical procedures associated with the medical practitioner, information describing performance of one or more medical procedures by the medical practitioner (e.g., procedure cards associated with the medical practitioner, usage patterns of pieces of medical equipment 160 or medical instruments by the medical practitioner, patterns of movement of the medical practitioner, etc.) times when the medical practitioner accessed the collaborative medical platform 140 from a client device 150, a location of a client device 150 used by the medical practitioner to access the collaborative medical platform 140, or any combination thereof. Metadata included in, or extracted from, the telemetry data or the video data is received by the practitioner prediction model in various embodiments. Example metadata from the telemetry data or video data includes a time when the telemetry data or video data was captured, a location where the telemetry data or video data was captured, a type of medical procedure during which the telemetry data or video data was captured, and any combination thereof.

The practitioner prediction model comprises a set of weights stored on a non-transitory computer readable storage medium. The analytics module 230 trains the practitioner prediction model by generating a training dataset including multiple training examples based on previously received telemetry data or video data and connections between one or more medical practitioners and the previously received telemetry data or video data. Different medical practitioners may be associated with telemetry data or video data included in different training examples. Each training example includes training telemetry data or training telemetry data and characteristics of a training user. Further, each training example has a label indicating whether the training medical practitioner is connected to the training telemetry data or to the training video data. For example, a label has a particular value in response to the training medical practitioner being connected to the training video data or to the training telemetry data has an alternative value in response to the training medical practitioner not being connected to the training telemetry data or to the training video data.

To train the practitioner prediction model, the analytics module 230 initializes the set of weights comprising the practitioner prediction model and applies the practitioner prediction model to multiple training examples of the training dataset. Applying the practitioner prediction model to multiple training examples updates one or more parameters (e.g., weights) comprising the practitioner prediction model. The parameters comprising the practitioner prediction model transform the input data—telemetry data or video data and characteristics of a medical practitioner—into a predicted probability of the telemetry data or the video data being connected to the medical practitioner. When applied to a training example, the practitioner prediction model generates the predicted probability of training telemetry data or training video data being connected to a training user based on the training telemetry data or the training video data and characteristics of the training user.

For each training example to which the practitioner prediction model is applied, the analytics module 230 generates a score comprising an error term based on the predicted probability of the training medical practitioner being associated with the training video data or with the training telemetry data and a label applied to the training example. The error term is larger when a difference between the predicted probability of the training medical practitioner being associated with the training video data or with the training telemetry data for the training example and the label applied to the training example is larger and is smaller when the difference between the predicted probability of the training medical practitioner being associated with the training video data or with the training telemetry data for the training example and the label applied to the training example is smaller. In various embodiments, the analytics module 230 generates the error term using a loss function based on a difference between the predicted probability of the training medical practitioner being associated with the training video data or with the training telemetry data for the training example and the label applied to the training example using a loss function. Example loss functions include a mean square error function, a mean absolute error, a hinge loss function, and a cross-entropy loss function.

The analytics module 230 backpropagates the error term to update the set of parameters comprising the practitioner prediction model and stops backpropagation in response to the error term, or to the loss function, satisfying one or more criteria. For example, the analytics module 230 backpropagates the error term through the practitioner prediction model to update parameters of the practitioner prediction model until the error term has less than a threshold value. For example, the analytics module 230 may apply gradient descent to update the set of parameters. The analytics module 230 stores the set of parameters comprising the practitioner prediction model on a non-transitory computer readable storage medium after stopping the backpropagation.

Various characteristics of a medical practitioner affect the probability of telemetry data or video data being connected to the medical practitioner determined by the practitioner prediction module. For example, characteristics indicating a medical practitioner did not access the collaborative medical platform 140 using a client device 150 during a time period corresponding to the telemetry data or the video data increases a probability of the video data or the telemetry data being connected to the medical practitioner, as a medical practitioner performing a medical procedure is unable to access the collaborative medical platform 140 using a client device 150. Similarly, a location associated with the medical practitioner matching a medical facility from which the telemetry data or video data was received increases the probability of the video data or the telemetry data being connected to the medical practitioner. As another example, characteristics of a medical practitioner indicating a location of the medical practitioner during a time interval corresponding to the telemetry data or video data matched a location where the telemetry data or video data was captured and indicating the medical practitioner did not access the collaborative medical platform 140 using a client device 150 during a time period corresponding to the telemetry data or the video data increases a probability of the telemetry data or video data being connected to the medical practitioner. As another example, patterns of movement of the medical practitioner during one or more medical procedures or usage patterns of pieces of medical equipment 160 during medical procedures stored in a user profile of the medical practitioner having higher measures of similarity to patterns of movement of a medical practitioner or usage patterns of a piece of medical equipment 160 identified from the telemetry data or video data increase a probability of the telemetry data or video data being connected to the medical practitioner. In an additional example, a type of medical procedure associated with a medical practitioner in a user profile matching a type of medical procedure the analytics module 230 determines for the telemetry data or video data increases a probability of the telemetry data or video data being connected to the medical practitioner. During the training process for the practitioner prediction model, relationships between characteristics of a medical practitioner and a probability of telemetry data or video data being connected to the medical practitioner are refined and are represented through parameters of the practitioner prediction model.

In various embodiments, the analytics module 230 applies the trained practitioner prediction model to the telemetry data or video data and each of a set of medical practitioners to generate a probability of each medical practitioner of the set being connected to the telemetry data or the video data. Each medical practitioner of the set has one or more specific characteristics in various embodiments. For example, the analytics module 230 identifies a medical facility from which telemetry data or video data was received from metadata included in the telemetry data or the video data and selects the set of medical practitioners as medical practitioners having a location matching the identified medical facility. As another example, the analytics module 230 determines a date corresponding to the telemetry data or the video data, such as from metadata included in the telemetry data or video data, and determines a medical facility from which the telemetry data or video data was received from metadata; the analytics module 230 identifies the set of medical practitioners as medical practitioners with locations on the determined date matching the determined medical facility. Selecting the set of medical practitioners limits a number of medical practitioners to which the practitioner prediction model is applied.

Alternatively, the analytics module 230 maintains a set of rules applied to characteristics of a medical practitioner and to telemetry data or video data to determine a probability of the medical practitioner being connected to the telemetry data or video data. In various embodiments, each rule identifies one or more characteristics of the medical practitioner and criteria for comparing the one or more characteristics to the telemetry data or video data. In response to comparing of one or more characteristics of the medical practitioner in a rule to the telemetry data or video data as specified by a rule indicating the one or more characteristics satisfy the rule, the analytics module 230 increases a probability of the medical practitioner being connected to the telemetry data or video data. For example, a rule identifies an indication the medical practitioner accessed the collaborative medical platform and a criterion that the indication was negative during a time interval corresponding to telemetry data or video data and criteria. In response to the indication the medical practitioner accessed the collaborative medical platform being negative during the time interval corresponding to the telemetry data or video data, the analytics module 230 increases a probability of the medical practitioner being connected to the telemetry data or video data. As another example, a rule identifies a location of the medical practitioner and a criterion that the location of the medical practitioner match a location identified by the telemetry data or video data. In response to the location of the medical practitioner matching the location identified by the telemetry data or video data, the analytics module 230 increases a probability of the medical practitioner being connected to the telemetry data or video data. For another example, a rule increases a probability of the medica practitioner being connected to the telemetry data or video data in response to a type of medical procedure associated with the telemetry data or video data matching a type of medical procedure associated with the medical practitioner. As another example, one or more rules compare patterns of movement of the medical practitioner during one or more medical procedures or usage patterns of pieces of medical equipment 160 during medical procedures stored in a user profile of the medical practitioner to patterns of movement of a medical practitioner or usage patterns of a piece of medical equipment 160 identified from the telemetry data or video data and increase a probability of the telemetry data or video data being connected to the medical practitioner if one or more patterns from the user profile have higher measures of similarity to one or more patterns from the telemetry data or video data.

Having characteristics satisfying a greater number of rules results in a higher probability of the medical practitioner having a connection to the telemetry data or video data. In some embodiments, the analytics module 230 decreases the probability of the medical practitioner having a connection to the telemetry data or video data in response to characteristics of the user not satisfying one or more criteria in a rule. However, in other embodiments, the analytics module 230 does not modify the probability of the medical practitioner having a connection to the telemetry data or video data in response to characteristics of the user not satisfying one or more criteria in a rule. The analytics module 230 applies the rules to characteristics maintained for multiple medical practitioners of the set to determine probabilities of different medical practitioners being connected to the telemetry data or video data.

Based on the probabilities determined for each medical practitioner of the set, the analytics module 230 selects a medical practitioner. For example, the analytics module 230 ranks the medical practitioners of the set based on their probabilities of being connected to the video data or the telemetry data and selects a medical practitioner having at least a threshold position in the ranking, such as a maximum position in the ranking. Alternatively, the analytics module 230 selects a medical practitioner having at least a threshold probability or having a maximum probability. In various embodiments, the analytics module 230 automatically stores a connection between the selected medical practitioner and the telemetry data or video data.

Alternatively, the analytics module 230 transmits a prompt to a client device 150 of the selected medical practitioner including descriptive information about the video data or the telemetry data and a request for the medical practitioner to confirm a connection between the selected medical practitioner and the video data or the telemetry data. The descriptive information about the video data or the telemetry data may be a portion of the video data, metadata associated with the video data or the telemetry data, or any combination thereof. For example, the analytics module 230 applies a classification model to the video data or the telemetry data that determines a type of medical procedure corresponding to the video data or to the telemetry data, and the descriptive information comprises the determined type of medical procedure. As another example, the descriptive information comprises the determined type of medical procedure, a location associated with the telemetry data or video data, and a time associated with the telemetry data or video data. Including descriptive information about the telemetry data or the video data in the prompt simplifies identification of the medical procedure for the selected medical practitioner to determine whether the selected medical practitioner is associated with the medical procedure during which the telemetry data or the video data was captured. Storing the connection between the selected medical practitioner and the telemetry data or video data in response to receiving the confirmation allows the selected medical practitioner to confirm that the selected medical practitioner performed, or was associated with, the medical procedure during which the telemetry data or video data was captured before the collaborative medical platform connects the selected medical practitioner to the video data or the telemetry data.

In response to receiving a confirmation from a client device 150 of the selected medical practitioner in response to the prompt, the analytics module 230 generates and stores a connection between an identifier of the selected medical practitioner and the telemetry data or video data in the connection graph store 255. The stored connection between the selected medical practitioner and the telemetry data or video data indicates that the selected medical practitioner is associated with the telemetry data or video data, so one or more metrics the analytics module 230 generates from the telemetry data or video data are associated with the selected medical practitioner. Similarly, recommendations for educational content or for reference content made by the practitioner education module 235 based on the telemetry data or the video data are presented to, or otherwise associated with, the selected medical practitioner because of the connection between the selected medical practitioner and the telemetry data or video data.

In some embodiments, in response to storing a connection between the selected medical practitioner and the telemetry data or video data, the collaborative medical platform 140 receives supplemental content about a medical procedure during which the telemetry data or video data was captured from the selected medical practitioner. The collaborative medical platform 140 stores the supplemental information from the selected medical practitioner along with the connection between the telemetry data or video data and the selected medical practitioner. In various embodiments, the analytics module 230 requests particular types of supplemental information from the selected medical practitioner for augmenting the video data or the telemetry data when storing the connection between eh selected medical practitioner and the telemetry data or video data.

In some embodiments, the analytics module 230 applies a generative model, such as a large language model (LLM), to metadata extracted from or determined from the telemetry data or the video data to generate one or more requests for supplemental data from the selected medical practitioner. The requests allow the analytics module 230 to identify specific types of supplemental information to receive from the selected medical practitioner. The generative model may be applied to information from a user profile of the selected medical practitioner, such as types of medical procedures performed by the medical practitioner, preferences for performing one or more types of medical procedures (e.g., procedure cards in a user profile for the selected medical practitioner), or other information associated with the selected medical practitioner by the collaborative medical platform 140 in conjunction with the telemetry data or video data to generate one or more requests tailored for the selected medical practitioner. In some embodiments, the generative model is applied to previously received supplemental information to generate additional requests that leverage supplemental information previously received in response to other prompts.

Alternatively, the analytics module 230 maintains a set of predefined requests that are presented to the selected medical practitioner. For example, the analytics module 230 maintains a sequence of predefined requests for supplemental information that are presented in a stored order to the selected medical practitioner. In various embodiments, the analytics module 230 maintains one or more forms that each include one or more requests for supplemental information that are presented to the selected medical practitioner after a connection is stored between the selected medical practitioner and video or telemetry data. Data received from the selected medical practitioner in response to one or more requests comprises the supplemental information in various embodiments.

For example, supplemental information includes notations or comments about the medical procedure provided by the selected medical practitioner. As another example, supplemental information comprises modifications to metadata that the analytics module 230 determined from the telemetry data or video data. In an additional example, supplemental information is a modification to a type of medical procedure during which the telemetry data or video data was captured. Supplemental information may be associated with one or more portions of the telemetry data or video data in various embodiments. Different portions of supplemental information may be associated with different portions of the telemetry data or video data.

As additional examples, supplemental information from the selected medical procedure includes a description of steps of the medical procedure. The description of a step may include notes from the selected medical practitioner describing positioning of pieces of medical equipment 160 for performing the step, relative positioning of medical instruments to each other for use by the selected medical practitioner during the step, one or more techniques used by the selected medical practitioner when performing the step, or other information describing how the selected medical practitioner performs the step of the medical procedure. Hence, the supplemental information may include preferences of the selected medical practitioner for performing one or more steps of the medical procedure.

The analytics module 230 subsequently stores the supplemental information in association with the selected medical practitioner and with the telemetry data or video data. For example, the analytics module 230 stores the supplemental information in a user profile of the selected medical practitioner along with the connection between the selected medical practitioner and the telemetry data or the video data. As further described above, a user profile for the selected medical practitioner may include procedure cards associated with different types of medical procedures, with procedure cards associated with a type of medical procedure describing practitioner-specific preferences for performing one or more steps of the type of medical procedure. The analytics module 230 updates one or more procedure cards associated with the type of medical procedure during which the telemetry data and the video data were captured based on the supplemental information in some embodiments, simplifying modification of procedure cards for the selected medical practitioner based on the supplemental information from the selected medical practitioner.

Additionally, in various embodiments, the analytics module 230 leverages the connection between the selected medical practitioner and the telemetry data or video data to generate configuration information for one or more pieces of medical equipment 160 in various embodiments. For example, the analytics module 230 trains or obtains one or more confirmation models that receive telemetry data or video data of a medical practitioner performing one or more medical procedures and that generate configuration information for one or more pieces of medical equipment 160 based on the telemetry data or video data. The analytics module 230 stores configuration information in a user profile of a user connected to the telemetry data to video data to which a configuration model was applied and stores the configuration information in association with a piece of medical equipment 160. In various embodiments, the configuration information is stored in a user profile for a medical practitioner in association with a type of medical procedure and with an identifier of the piece of medical equipment 160. The configuration information may be provided to an associated piece of medical equipment 160 from the collaborative medical platform 140 to configure the piece of medical equipment 160 for use by the medical practitioner for whom the configuration information was stored. This allows the collaborative medical platform 140 to subsequently automatically configure a piece of medical equipment 160 for use by a medical practitioner.

By selecting a medical practitioner associated with received telemetry data or video data, as further described above, the analytics module 230 increases an amount of information connected to the selected medical practitioner about operation or use of one or more pieces of medical equipment, allowing more accurate learning of usage patterns of pieces of medical equipment by one or more medical practitioners. Selecting the medical practitioner connected to telemetry data or video data, as further described above, enables the analytics module 230 to connect additional telemetry data or video data that includes use of one or more pieces of medical equipment 160 with the selected medical practitioner, providing more interactions between the selected medical practitioner and one or more pieces of medical equipment 160. Increasing information about interactions between a medical practitioner and one or more pieces of medical equipment allows the collaborative medical platform 140 to more accurately generate configuration information for automatically configuring a piece of medical equipment for use by the medical practitioner by.

However, in response to receiving a rejection from the selected medical practitioner in response to the prompt, the analytics module 230 does not store a connection between the medical practitioner and the telemetry data or video data. In response to receiving the rejection, the analytics module 230 selects an alternative medical practitioner for the telemetry data or video data, as further described above. For example, the analytics module 230 ranks the medical practitioners of the set based on their corresponding probabilities of having a connection to the telemetry data or video data and selects an alternative medical practitioner having at least a threshold position in the ranking. Additionally, the analytics module 230 may generate an additional training example for the practitioner prediction model based on a received response to a prompt, such an additional training example includes the telemetry data or video data, characteristics of the selected medical practitioner, and a label indicating the selected medical practitioner's response to the prompt. The analytics module 230 may subsequently apply the practitioner prediction model to the additional training example to refine one or more parameters of the practitioner prediction model, as further described above.

The practitioner education module 235 manages and stores training data for medical practitioners associated with medical procedures. In various embodiments, the training data comprises educational content including descriptive information about a medical procedure or about a portion of a medical procedure. Example educational content includes training videos relating to performing medical procedures, articles about performing medical procedures, articles or videos about using one or more pieces of medical equipment 160 in a medical procedure, articles or videos about using one or more medical instruments in a medical procedure, best practices for a medical procedure, training manuals for medical procedures, instructional material for one or more medical instruments used in a medical procedure, digital training modules, webinars, audio data about a medical procedure (e.g., a podcast about a medical procedure) or other information for training medical practitioners in relation to medical procedures.

In various embodiments, educational content also includes configuration data or configuration instructions for one or more pieces of medical equipment 160 used in one or more medical procedures. For example, educational content includes a set of configuration instructions for configuring or for calibrating a robotic arm or other piece of medical equipment 160 for use in a medical procedure. Configuration instructions may include one or more settings for the piece of medical equipment 160. Example settings include: one or more limiting values for an amount of force applied by a piece of medical equipment 160, one or more limiting values for a range of motion of a piece of medical equipment 160, one or more limiting values for an amount of energy supplied by a piece of medical equipment 160, an identifier of a mode of operation for a piece of medical equipment 160, or values for one or more other settings of a piece of medical equipment 160. As another example, educational content comprises a set of instructions that, when executed by a piece of medical equipment 160, cause the piece of medical equipment 160 to perform a sequence of actions for calibration. Certain educational content may be executable by a piece of medical equipment 160 to modify values of one or more settings of the piece of medical equipment 160 or a mode of operation of the piece of medical equipment 160, allowing automatic modification of one or more settings of the piece of medical equipment 160 via the educational content item without a medical practitioner manually specifying values of settings of the piece of medical equipment 160.

The practitioner education module 235 stores educational content as different educational content items, with each educational content item comprising a discrete portion of content, such as a file. Each educational content item has one or more attributes providing descriptive information about the educational content item. For example, an attribute of an educational content item identifies one or more types of medical procedures associated with the educational content item, allowing identification of educational content items corresponding to different types of medical procedures. Other example attributes of an educational content item include: one or more medical practitioners associated with the educational content item (e.g., a medical practitioner who performed a medical procedure associated with the educational content item, a medical practitioner who created the educational content item), a location associated with the educational content item (e.g., a geographic location, a specific medical facility), a time associated with the educational content item (e.g., a time when the educational content item was created), identifiers of one or more pieces of medical equipment 160 associated with the educational content item, identifiers of one or more medical instruments used in a medical procedure associated with the educational content item, a format of the educational content item (e.g., audio, video, text), or other information describing the educational content item. Educational content items may be locally stored by the collaborative medical platform 140 (e.g., in the video library 250 or another storage device) or retrieved from one or more third-party servers 170 in various embodiments.

One or more educational content items may comprise reference cases, which are medical cases that a medical practitioner who performed a completed medical procedure in a medical case selected to be available to other medical practitioners. For a reference case, the practitioner education module 235 stores video data, telemetry data from medical equipment 160, or other data captured by the collaborative medical platform 140 during performance of the completed medical procedure. In various embodiments, a reference case includes a content feed including comments or other data obtained by the collaborative medical platform 140 from contributors during the completed medical procedure. For a reference case, the practitioner education module 235 pseudonymizes patient data to prevent the reference case from including patient data capable of being attributed to a specific patient. In some embodiments, the pseudonymized patent data in a reference case identifies ranges for one or more types of patent data to maintain relevant information about a patent on whom the completed medical procedure was performed for another medical practitioner while preventing identification of a specific patient on whom the completed medical procedure was performed.

Each educational content item is associated with one or more baseline criteria. Different baseline criteria specify values for metrics from performing a medical procedure, settings for a piece of medical equipment 160 used for a medical procedure, movement patterns of a piece of medical equipment 160 during a medical procedure, patterns of telemetry data obtained during a medical procedure, movement patterns of a medical practitioner during a medical procedure, or other descriptive information about performing a medical procedure. A baseline criterion specifies a standardized value for a metric, a standardized technique or approach used in a medical procedure, or other standardized value or technique related to a medical procedure. The practitioner education module 235 maintains one or more baseline criteria for different medical procedures, so different educational content items correspond to different medical procedures. An attribute of an educational content item comprises an identifier of a type of medical procedure to indicate the educational content item and its associated baseline criteria correspond to the type of medical procedure. This allows the practitioner education module 235 to identify different baseline criteria for different types of medical procedures.

In various embodiments, one or more medical practitioners input baseline criteria for a medical procedure to the practitioner education module 235. For example, a group of medical practitioners reach a consensus on values of metrics, patterns of telemetry data, patterns of movement, or values of other information describing performance of a medical procedure. A medical practitioner of the group inputs the agreed-upon baseline criteria to the practitioner education module 235 for storage in association with an educational content item. The group of medical practitioners may be associated with a particular medical facility (e.g., a hospital, a clinic), to provide facility-specific baseline criteria. The practitioner education module 235 stores an identifier of a medical facility as an attribute of an educational content item associated with the facility-specific baseline criteria to indicate baseline criteria associated with a specific medical facility. Additionally or alternatively, the group of medical practitioners who determined baseline criteria are not associated with a particular medical facility, but correspond to a larger organization or standards body, so the baseline criteria for the medical procedure are applicable across various medical facilities. The practitioner education module 235 may store facility-specific baseline criteria and more generally applicable baseline criteria as different attributes of an educational content item in various embodiments. This allows augmentation of more generally applicable baseline criteria associated with an educational content item with facility-specific baseline criteria.

In various embodiments, the practitioner education module 235 generates one or more baseline criteria associated with an educational content item by applying one or more trained machine learned models to metrics generated for multiple medical cases in which a type of medical procedure was performed by the analytics module 235. In various embodiments, the one or more trained machined learned models are also applied to telemetry data or video data captured by the telepresence module 225 during medical cases where the type of medical procedure was performed. For example, a machine learned model detects patterns in telemetry data captured during medical procedures of a specific type occurring in medical cases for which a specific value of a generated metric was generated or for which a value of a generated metric is within a range of values. The specific value of a generated metric or a range of values of the generated metric may correspond to one or more specific patient outcomes. For example, the specific value or range of values identifies successful patient outcomes for the type of medical procedure. One or more patterns of telemetry data detected with at least a threshold frequency in medical procedures occurring in medical cases for which the generated metric has the specific value or has a value within a specified range are stored as baseline criteria for an educational content item associated with the specific type of medical procedure in various embodiments.

For example, application of a machine learned model to telemetry data identifies a specific sequence of movement of a piece of medical equipment 160 detected with at least a threshold frequency in completed medical procedures of the specific type performed in medical cases a metric corresponding to a positive outcome are stored as baseline criteria for an educational content item corresponding to movement of the piece of medical equipment 160 for the specific type of medical procedure. Telemetry data describing the specific sequence of movement of the piece of medical equipment 160 may be stored in the educational content item to specify limits of movement of the piece of medical equipment 160 during the specific type of medical procedure or to specify limits on force applied by the piece of medical equipment 160 during the specific type of medical procedure. As another example, captured telemetry data includes positional data for a piece of medical equipment 160 during occurrences of the type of medical procedure occurring in medical cases with one or more metrics correlated with positive outcomes for a patient. The practitioner education module 235 stores an educational content item associated with the type of medical procedure having the positional data in the captured telemetry data as a baseline criterion. This allows the practitioner education module 235 to dynamically generate an educational content item and associated baseline criteria for a type of medical procedure based on telemetry data captured during performance of the type of medical procedure over time, simplifying generation of educational content items for various medical procedures.

In other examples of generating educational content item from telemetry data, telemetry data from a piece of medical equipment includes bimanual dexterity of the medical practitioner during a medical procedure, with the bimanual dexterity information stored in an educational content item as baseline criteria in response to determining the medical procedure had a positive outcome. In various embodiments, the generated educational content item includes data for accessing a simulator for the piece of medical equipment 160 used during the medical procedure (e.g., an identifier of a simulator, one or more exercises or techniques to perform on the identified simulator, etc.) to further refine use of the piece of medical equipment 160 in response to telemetry data from a medical practitioner during a medical procedure including bimanual dexterity information deviating from the baseline criteria of the educational content item by at least a threshold amount. As another example, telemetry data from a piece of medical equipment 160 includes tissue tension for a patient during a medical procedure, with the tissue tension stored in an educational content item as baseline criteria in response to the practitioner education module 235 determining the medical procedure had a positive outcome. The generated educational content item may include data for accessing a simulator for the piece of medical equipment 160 used during the medical procedure (e.g., an identifier of a simulator, one or more exercises or techniques to perform on the identified simulator, etc.) to further refine use of the piece of medical equipment 160 in response to telemetry data from a medical practitioner during a medical procedure including bimanual dexterity information deviating from the baseline criteria of the educational content item by at least a threshold amount.

Additionally or alternatively, the practitioner education module 235 applies one or more machine learned models to video data captured during performances of a specific type of medical procedure during prior medical cases to identify different pieces of medical equipment 160 used during the specific type of medical procedure, movement of different pieces of medical equipment 160 during the specific type of medical procedure, movement of the medical practitioner performing the specific type of medical procedure, or other information about performing the specific type of medical procedure. As further described above, applying a machine learning model to video data of prior performances of the specific type of medical procedure detects patterns of movement of the medical practitioner or of a piece of medical equipment 160 during performance of the specific type of medical procedure. A pattern or movement detected with at least a threshold frequency in video data of completed medical procedures of the specific type performed in medical cases having a metric corresponding to a positive outcome are stored as baseline criteria for one or more educational content items associated with the specific type of medical procedure. Such an educational content item associated with the type of medical procedure and baseline criteria describing a pattern of movement includes positional data or other data describing movement or positioning of the medical practitioner or for a piece of medical equipment 160 during the specific type of medical procedure for subsequent reference. Other information, such as depth perception data, proximity of a piece of medical equipment 160 to a structure of the patient, an angle of transection of a structure of a patient by a piece of medical equipment 160, path length of a piece of medical equipment 160, tissue tension, bimanual dexterity of a medical practitioner, or other data may be determined from video data by the practitioner education module 235 and stored as baseline criteria in response to being determined from video data of a medical procedure with a threshold frequency or in response to being determined from video data of a medical procedure having a metric corresponding to a positive outcome. This allows the practitioner education module 235 to determine baseline criteria for a type of medical procedure based on video data of one or more medical procedures.

To select an educational content item for a medical practitioner, the practitioner education module 235 compares data describing performance of a medical procedure performed by the medical practitioner to baseline criteria associated with various educational content items. In various embodiments, after the medical practitioner completes the medical procedure, the practitioner education module 235 identifies educational content items associated with a type of the medical procedure and compares obtained information describing the medical procedure to one or more baseline criteria associated with the identified educational content items. For example, the practitioner education module 235 compares a metric generated for the medical procedure by the analytics module 230 from captured telemetry data, video data, or other data to a baseline criterion associated with educational content items associated with the type of the medical procedure. In response to the metric differing from the baseline criterion associated with an educational content item by at least a threshold amount, or otherwise failing to satisfy the baseline criterion, the practitioner education module 235 selects the educational content item associated with the baseline criterion for presentation to the medical practitioner. For example, in response to determining an amount of time for the medical practitioner to complete a medical procedure exceeds an average amount of time to complete the type of medical procedure or exceeds a baseline amount of time to complete the type of medical procedure, and selects one or more educational content items associated with the type of medical procedure and associated with baseline criteria specifying an amount of time to complete the type of medical procedure. In some embodiments, the practitioner education module 235 selects one or more educational content items associated with a type of medical procedure and associated with baseline criteria from which a metric determined for the medical procedure differs by at least a threshold amount, allowing the practitioner education module 235 to account for a specific amount of variance between a determined metric and a baseline criterion when selecting an educational content item.

Alternatively or additionally, the practitioner education module 235 compares one or more patterns or data detected within telemetry data captured during performance of the medical procedure to baseline criteria associated with educational content items. The practitioner education module 235 selects an educational content item associated with the type of the medical procedure and associated with a baseline criterion specifying a pattern of telemetry data differing from the captured telemetry data by at least a threshold amount. For example, telemetry data includes depth perception data during the medical procedure, and the practitioner education module 235 selects an educational content item associated with the type of the medical procedure and associated with depth perception data differing from the captured depth perception data by at least a threshold amount. The selected educational content item includes information for accessing a simulator for the piece of medical equipment 160 (e.g., an identifier of a simulator, one or more exercises or techniques to perform on the identified simulator, etc.) to be accessed by the medical practitioner in some embodiments. In another example, telemetry data includes tissue tension data captured during the medical procedure, and the practitioner education module 235 selects an educational content item associated with the type of the medical procedure and associated with tissue tension data differing from the captured tissue tension data by at least a threshold amount. The selected educational content item includes information for accessing a simulator for the piece of medical equipment 160 (e.g., an identifier of a simulator, one or more exercises or techniques to perform on the identified simulator, etc.) to be accessed by the medical practitioner in various embodiments.

Telemetry data from one or more sensors (e.g., sensors included in a piece of medical equipment 160) may also describe movement or positioning of medical equipment 160 or medical instruments during a medical procedure, and the practitioner education module 235 selects an educational content item including baseline criteria from which the movement of a piece of medical equipment or the positioning of a medical instrument in the telemetry data deviates by at least a threshold amount. For example, telemetry data includes a path length of a piece of medical equipment 160 during the medical procedure, and the practitioner education module 235 selects an educational content item associated with the type of medical procedure and including baseline criteria specifying a path length of the piece of medical equipment 160 from which the path length in the captured telemetry data deviated by at least a threshold amount. As another example, telemetry data includes positional data of a piece of medical equipment 160 during the medical procedure, and the practitioner education module 235 selects an educational content item associated with the type of medical procedure and including baseline criteria specifying positional data of the piece of medical equipment 160 from which the positional data of the piece of medical equipment 160 in the captured telemetry data deviated by at least a threshold amount. In an additional example, telemetry data includes bimanual dexterity data of the medical practitioner during the medical procedure, and the practitioner education module 235 selects an educational content item associated with the type of medical procedure and including baseline criteria specifying bimanual dexterity data of the piece of medical equipment 160 from which the bimanual dexterity data in the captured telemetry data deviated by at least a threshold amount. In the preceding examples, an educational content item selected based on the telemetry data includes information for accessing a simulator (e.g., through the collaborative medical platform 140) associated with the piece of medical equipment 160 corresponding to the telemetry data, providing the medical practitioner with increased interaction with the piece of medical equipment 160. Further, an educational content item selected based on deviation in positional data of a piece of medical equipment 160 from baseline criteria may include one or more of: a training video associated with the piece of medical equipment 160 and describing operation of the piece of medical equipment, audio data describing operation of the piece of medical equipment 160, and information for accessing a simulator for the piece of medical equipment 160 (e.g., an identifier of a simulator, one or more exercises or techniques to perform on the identified simulator, etc.). In some embodiments, the captured telemetry data describes a usage pattern of a piece of medical equipment 160 during the medical procedure. In response to determining the usage pattern of the piece of medical equipment 160 deviates from a baseline usage pattern of the piece of medical equipment in an educational content item, the practitioner education module 235 selects the educational content item, which may include benchmarking data describing a cost of the medical procedure based on the usage pattern and information about alternative usage patterns of the piece of medical equipment 160 to reduce the cost or information describing recommended usage patterns of the piece of medical equipment 160 or use of alternative pieces of medical equipment 160 in the type of medical procedure.

In various embodiments, the practitioner education module 235 compares one or more data identified from video data captured during performance of the medical procedure to baseline criteria associated with educational content items to select one or more educational content items for a medical practitioner. The practitioner education module 235 selects an educational content item associated with the type of the medical procedure and associated with a baseline criterion specifying specific data differing from the data identified from the captured video by at least a threshold amount. For example, the practitioner education module 235 obtains depth perception data during the medical procedure from video data of the medical procedure and selects an educational content item associated with the type of the medical procedure and associated with depth perception data differing from the depth perception data determined from the video data of the medical procedure by at least a threshold amount. The selected educational content item includes information for accessing a simulator for the piece of medical equipment 160 (e.g., an identifier of a simulator, one or more exercises or techniques to perform on the identified simulator, etc.) to be accessed by the medical practitioner in some embodiments. In another example, the practitioner education module 235 determines tissue tension data during the medical procedure and selects an educational content item associated with the type of the medical procedure and associated with tissue tension data differing from the tissue tension data determined from the video data by at least a threshold amount. The selected educational content item includes information for accessing a simulator for the piece of medical equipment 160 (e.g., an identifier of a simulator, one or more exercises or techniques to perform on the identified simulator, etc.) to be accessed by the medical practitioner in various embodiments.

The practitioner education module 235 determines movement or positioning of medical equipment 160 or medical instruments during a medical procedure from video data of the medical procedure through one or more computer vision models or other models in various embodiments. Based on the movement or positioning information obtained from the video data, the practitioner education module 235 selects an educational content item including baseline criteria from which the movement of a piece of medical equipment or the positioning of a medical instrument in the telemetry data deviates by at least a threshold amount. For example, the practitioner education module 235 determines a path length of a piece of medical equipment 160 during the medical procedure from video data of the medical procedure, and the practitioner education module 235 selects an educational content item associated with the type of medical procedure and including baseline criteria specifying a path length of the piece of medical equipment 160 from which the path length from the video data deviated by at least a threshold amount. As another example, the practitioner education module 235 determines positional data of a piece of medical equipment 160 during the medical procedure from video of the medical procedure, and the practitioner education module 235 selects an educational content item associated with the type of medical procedure and including baseline criteria specifying positional data of the piece of medical equipment 160 from which the positional data of the piece of medical equipment 160 from the video data deviated by at least a threshold amount. In an additional example, the practitioner education module 235 determines bimanual dexterity data of the medical practitioner during the medical procedure from the video data, and the practitioner education module 235 selects an educational content item associated with the type of medical procedure and including baseline criteria specifying bimanual dexterity data of the piece of medical equipment 160 from which the bimanual dexterity data determined from the video data deviated by at least a threshold amount. In the preceding examples, an educational content item selected based on the telemetry data includes information for accessing a simulator (e.g., an identifier of a simulator, one or more exercises or techniques to perform on the identified simulator, etc.) associated with the piece of medical equipment 160 corresponding to the telemetry data, providing the medical practitioner with increased interaction with the piece of medical equipment. Further, an educational content item selected based on deviation in positional data of a piece of medical equipment 160 from baseline criteria may include one or more of: a training video associated with the piece of medical equipment 160 and describing operation of the piece of medical equipment, audio data describing operation of the piece of medical equipment 160, and information for accessing a simulator for the piece of medical equipment 160 (e.g., an identifier of a simulator, one or more exercises or techniques to perform on the identified simulator, etc.).

In some embodiments, the practitioner education module 235 determines an angle at which a structure of a patient (e.g., an organ of the patient) is transected by a piece of medical equipment 160 (or by a medical instrument) during the medical practitioner form the video data of the medical procedure. The practitioner education module 235 selects an educational content item associated with the type of medical procedure and including an angle for transecting the structure of the patient from which the determined angle from the video data of the medical procedure deviated by at least a threshold amount. An educational content item selected based on deviation of a determined angle of transection of a structure of the patient from a baseline angle of transection may include content describing usage of the piece of medical equipment 160 (or medical instrument) transecting the structure of the patient during the medical procedure or content describing correlations between the angle of transection of the structure of the patient and one or more outcomes of the medical procedure (e.g., information depicting correlation between certain angles of transecting the structure of the patient and positive outcomes of the medical procedure or correlations between angles of transecting the structure of the patient and negative outcomes of the medical procedure). As another example, the practitioner education module 235 determines a proximity of a piece of medical equipment 160 (or a medical instrument) to one or more critical structures (e.g., an organ, a bone, an artery) of the patient during the medical procedure from video data of the medical procedure. The practitioner education module 235 selects an educational content item associated with the type of medical procedure and including a baseline proximity of the piece of medical equipment 160 (or medical instrument) from the critical structure of the patient from which the proximity of the piece of medical equipment 160 (or medical instrument) from the video data deviated by at least a threshold amount. In various embodiments, the educational content item with the baseline proximity to the critical structure of the patient includes content describing use of energy devices during medical procedures, which may include interactive content (e.g., content with questions to be answered by the medical practitioner), video or audio content describing use of energy devices during medical procedures, or other descriptive information about use of energy devices during medical procedures.

Further, the practitioner education module 235 may determine a usage pattern of a piece of medical equipment 160 during the medical procedure from video data of the medical procedure. In response to determining the usage pattern of the piece of medical equipment 160 deviates from a baseline usage pattern of the piece of medical equipment in an educational content item, the practitioner education module 235 selects the educational content item. The selected educational content item may include benchmarking data describing a cost of the medical procedure based on the usage pattern and information about alternative usage patterns of the piece of medical equipment 160 to reduce the cost. As another example, the selected educational content item may include information describing recommended usage patterns of the piece of medical equipment 160 or use of alternative pieces of medical equipment 160 in the type of medical procedure.

In another example, the practitioner education module 235 compares one or more patterns of movement (e.g., movement of a piece of medical equipment 160, movement of a portion of the medical practitioner) detected within video data captured during the medical procedure to baseline criteria including a pattern of movement for the type of the medical procedure and selects an educational content item for the medical practitioner associated with a baseline criterion specifying a pattern of movement from which the detected pattern of movement differs by at least a threshold amount. Hence, the practitioner education module 235 may use data (e.g., telemetry data or video data) captured during performance of a medical procedure to determine when to select an educational content item for the medical practitioner. Different detected patterns within telemetry data or video data captured during performance of a medical procedure may be compared to different educational content items each associated with different baseline criteria. This allows the practitioner education module 235 to select an educational content item for a medical practitioner based on specific portions of the medical procedure that deviated from a corresponding baseline criterion based on telemetry data or video data captured during performance of a medical procedure, allowing tailoring of educational content item selection to specific portions of the medical procedure.

The practitioner education module 235 may apply one or more trained machine learning models to data describing performance of a medical procedure performed by the medical practitioner and to attributes of educational content items, such as educational content items associated with a type of the medical procedure, to select one or more educational content items for presentation to the medical practitioner. Example attributes of an educational content item include: a type of medical procedure associated with the reference content item, one or more medical practitioners associated with the reference content item, a location where the medical procedure was performed (e.g., a geographic location, an identifier of a medical facility), a format of the reference content item (e.g., text data, audio data, video data, etc.), feedback about the reference content item from or more medical practitioners (e.g., a rating, an amount of positive feedback received for the reference content item, etc.), or other descriptive information. The practitioner education module 235 trains one or more machine-learning models to select one or more educational content items for a medical practitioner based on attributes of educational content items and characteristics of the medical practitioner in various embodiments. Example machine learning models include regression models, support vector machines, naïve Bayes, decision trees, k nearest neighbors, random forest, boosting algorithms, k-means, and hierarchical clustering. The machine learning models may also include neural networks, such as perceptrons, multilayer perceptrons, convolutional neural networks, recurrent neural networks, sequence-to-sequence models, generative adversarial networks, or transformers, while other types of machine learning models may additionally or alternatively be trained or applied by the practitioner education module 235 in various embodiments.

For example, to train a machine learning model to select one or more educational content items, the practitioner education module 235 generates a set of training examples, with each training example including data describing performance of a medical procedure performed by a medical practitioner and attributes of an educational content item and having a label indicating whether the medical practitioner in the training example accessed the educational content item included in the training example (or indicating whether the medical practitioner in the training example provided positive feedback for the educational content item included in the training example).

Applying the machine learning model to a training example generates a predicted likelihood of the medical practitioner in the training example accessing the educational content item in the training example (or a predicted likelihood of the medical practitioner in the training example providing positive feedback for the educational content item included in the training example). For each training example to which the practitioner education module 235 applies the machine learning model, the practitioner education module 235 generates a score for the machine learning model comprising an error term based on the label applied to the training example and the predicted likelihood of the medical practitioner in the training example accessing the educational content item in the training example (or a predicted likelihood of the medical practitioner in the training example providing positive feedback for the educational content item included in the training example). The error term, and accordingly the score, is larger when a difference between the label applied to the training example and the predicted likelihood of the medical practitioner in the training example accessing the educational content item in the training example (or the predicted likelihood of the medical practitioner in the training example providing positive feedback for the educational content item included in the training example) is larger and is smaller when the difference between label applied to the training example and the predicted likelihood of the medical practitioner in the training example accessing the educational content item in the training example (or the predicted likelihood of the medical practitioner in the training example providing positive feedback for the educational content item included in the training example) is smaller. In various embodiments, the practitioner education module 235 generates the score for the machine learning model applied to a training example using a loss function based on the difference between the label applied to the training example and the predicted likelihood of the medical practitioner in the training example accessing the educational content item in the training example (or the predicted likelihood of the medical practitioner in the training example providing positive feedback for the educational content item included in the training example). Example loss functions include a mean square error function, a mean absolute error function, a hinge loss function, and a cross-entropy loss function.

The practitioner education module 235 backpropagates the error term to update a set of parameters comprising the machine learning model and stops backpropagation in response to the score, or to a loss function, satisfying one or more criteria. For example, the practitioner education module 235 backpropagates the score for the machine learning model through the layers of the machine learning model to update parameters of the machine learning model until the score has less than a threshold value. For example, the practitioner education module 235 uses gradient descent to update the set of parameters comprising the machine learning model. The practitioner education module 235 stores the trained machine learning model for application to data describing performance of a medical procedure performed by the medical practitioner and to attributes of one or more educational content items. In some embodiments, the practitioner education module 235 trains and maintains different machine learning models that each use different combinations of attributes of an educational content item and data describing performance of a medical procedure performed by the medical practitioner.

Alternatively or additionally, one or more machine learning models applied by the practitioner education module 235 to select an educational content item are nearest neighbor models applied to embeddings corresponding to educational content items and to characteristics of the medical practitioner, including data describing performance of a medical procedure performed by the medical practitioner. As further described above, attributes of an educational content item include: a type of medical procedure associated with the reference content item, one or more medical practitioners associated with the reference content item, a location where the medical procedure was performed (e.g., a geographic location, an identifier of a medical facility), a format of the reference content item (e.g., text data, audio data, video data, etc.), feedback about the reference content item from or more medical practitioners (e.g., a rating, an amount of positive feedback received for the reference content item, etc.), or other descriptive information. Example characteristics of a medical practitioner include: an area of specialization of the medical practitioner, types of prior medical procedures performed by the medical practitioner, medical procedures scheduled to be performed by the medical practitioner, a location where the medical practitioner performs medical procedures (e.g., a geographic location, an identifier of a medical facility, etc.), collaborators connected to the medical practitioner via the connection graph, or other descriptive information about the medical practitioner, as well as data further described above describing performance of a medical procedure by the medical practitioner.

In some embodiments, the practitioner education module 235 applies a nearest neighbor model to an embedding of the medical practitioner that determines a distance (or a measure of similarity) in a latent space between the embedding of the medical practitioner and embeddings for various educational content items. For example, the nearest neighbor model determines a Euclidean distance between the embedding of the medical practitioner and embeddings for educational content items. Based on the distances, the nearest neighbor model ranks educational content items by the distances (or measures of similarity) of their corresponding embeddings to the embedding of the medical practitioner and selects one or more educational content items having a threshold position in the ranking, so the selected one or more educational content items have embeddings nearest to the embedding of the medical practitioner. Alternatively, the nearest neighbor model selects one or more educational content items having less than a threshold distance from the embedding for the medical practitioner. Alternatively, the practitioner education module 235 generates an embedding for a medical procedure performed by the medical practitioner and selects one or more educational content items based on distances between the embedding for the medical procedure and embeddings for educational content items, as further described above.

Further, in some embodiments, the practitioner education module 235 generates embeddings for different medical practitioners based on characteristics of the medical practitioners, as further described above. The practitioner education module 235 determines distances between an embedding for a medical practitioner and embeddings for additional medical practitioners. For example, the practitioner education module 235 determines Euclidean distances between the embedding for the medical practitioner and embeddings for multiple additional medical practitioners. Based on the distances (or measure of similarity), the practitioner education module 235 selects a set of additional medical practitioners. For example, the practitioner education module 235 selects additional medical practitioners with embeddings within a threshold distance of the embedding of the medical practitioner. As another example, the practitioner education module 235 ranks additional medical practitioners based on distances between their embeddings and the embedding of the medical practitioner and selects additional medical practitioners having at least a threshold position in the ranking. The practitioner education module 235 selects one or more educational content items presented to one or more of the selected additional medical practitioners for presentation to the medical practitioner. Such embodiments allow the practitioner education module 235 to leverage similarity between various medical practitioners to select educational content items for presentation to the medical practitioner.

In various embodiments, the practitioner education module 235 determines one or more baseline criteria for educational content items by applying one or more clustering models to one or more attributes of medical cases in which a specific type of medical procedure was performed. Attributes of a medical case include one or more metrics generated for the medical case by the analytics module 230, telemetry data captured during performance of the medical procedure in the medical case, video data captured during performance of the medical procedure in the medical case, or other descriptive information about the medical case. Based on attributes of a medical case, the practitioner education module 235 generates an embedding for the medical case. The practitioner education module 235 applies a clustering model to embeddings for different medical cases in which the specific type of medical procedure was performed to generate different clusters of case where a specific type of medical procedure was performed. Different clusters are represented in a latent space including the embeddings for medical cases by different centroids, with a cluster including medical cases having embeddings within a threshold distance of the cluster's centroid. In some embodiments, the practitioner education module 235 applies a k-means clustering model to embeddings for different medical cases in which the specific type of medical procedure was performed. Using k-means clustering causes a medical case in which the specific type of medical procedure was performed to be included in a cluster based on distances between the embedding for the medical case and centroids for different clusters. The medical case in which the specific type of medical procedure was performed is included in a cluster with a centroid having a minimum distance from the embedding for the medical case. Centroids of clusters are iteratively updated based on embeddings for medical cases in which the specific type of medical procedure was performed included in various clusters until one or more criteria are satisfied. This results in a specific number of clusters, each including medical cases in which the specific type of medical procedure was performed having similar embeddings.

The practitioner education module 235 may identify baseline criteria based on medical cases included in one or more clusters. For example, a cluster of cases in which the specific type of medical procedure was performed corresponds to positive outcomes for the specific type of medical procedure, while an alternative cluster corresponds to negative outcomes for the specific type of medical procedure. Based on captured telemetry data or video data during performance of the specific type of medical procedure in an additional case, the practitioner education module 235 generates an embedding for the additional case and determines a cluster including the additional case based on the centroids of the clusters and the embedding for the additional case. In response to determining the additional case is included in the alternative cluster corresponding to negative outcomes, the practitioner education module 235 selects one or more educational content items for presentation to the medical practitioner performing the specific type of medical procedure during the additional case. The practitioner education module 235 compares telemetry data or video data captured during performance of the specific type of medical procedure in the additional medical case to telemetry data or video data associated with baseline criteria of educational content items associated with the specific type of medical procedure and selects one or more educational content associated with the specific type of the medical procedure and having baseline criteria specifying telemetry data or video data differing from the telemetry data or video data captured during performance of the specific type of medical procedure by at least a threshold amount.

Alternatively, the practitioner education module 235 selects an educational content item for a medical case in response to determining the embedding for the medical case is not included in a particular cluster. As an example, the practitioner education module 235 selects an educational content item for a medical case in response to determining an embedding for the medical case is greater than a threshold distance from a centroid of a particular cluster of medical cases. This may indicate that the medical case has characteristics that deviate at least a threshold amount from characteristics of other medical cases with positive patient outcomes in which the type of medical procedure was performed. As further described above, the practitioner education module 235 may select an educational content item associated with a specific type of medical procedure being performed in the medical case and having baseline criteria including telemetry data or video data differing from the telemetry data or video data captured during performance of the medical procedure in the medical case by at least a threshold amount.

When generating clusters of medical cases based on corresponding embeddings, the practitioner education module 235 may identify medical cases included in a particular cluster as reference cases for educational content items for a corresponding type of medical procedure. For example, in response to the practitioner education module 235 including a medical case in a specific cluster associated with positive outcomes, the practitioner education module 235 communicates a prompt to a medical practitioner associated with the medical case to generate a reference case based on the medical case. In response to receiving authorization from the medical practitioner to generate the reference case from the medical case, the practitioner education module 235 pseudonymizes patient data in the medical case and stores the pseudonymized patent data, video data captured during performance of the medical procedure, telemetry data captured during performance of the medical procedure, and one or more metrics generated for the medical procedure as an educational content item for the type of the medical procedure. One or more patterns determined from telemetry data or video data, or one or more generated metrics, are stored as baseline criteria associated with the educational content item. This simplifies creation of educational content items for a type of medical procedure by leveraging data captured by the collaborative medical platform 140 during performance of medical procedures to generate educational content items for subsequent reference about the medical procedure.

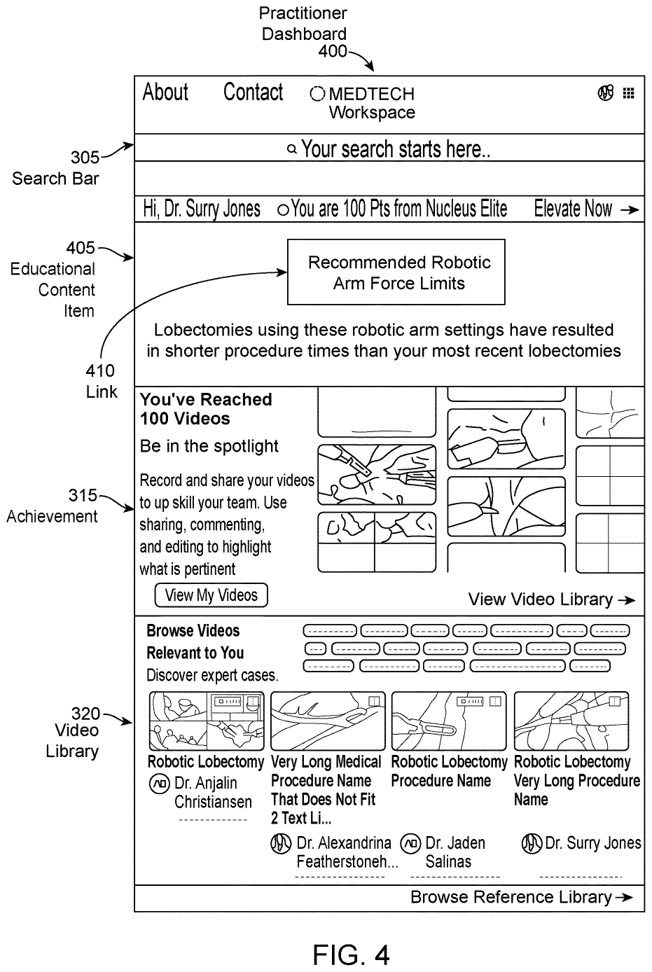

In various embodiments, an educational content item selected for a medical practitioner based on performance of a medical procedure by the medical practitioner is presented to the medical practitioner during a postprocedural stage. Presenting an educational content item to a medical practitioner during the postprocedural stage allows review of the educational content item after completion of a medical procedure. The practitioner education module 235 generates one or more interfaces that identify a selected educational content item to a medical practitioner. For example, the practitioner education module 235 includes information identifying a selected educational content item in a practitioner dashboard presented to the medical practitioner, such as a practitioner dashboard further described below in conjunction with FIG. 4. In various embodiments, information identifying a selected educational content item includes a link that, when selected by the medical practitioner, retrieves the selected educational content item for presentation. Alternatively, the practitioner education module 235 presents information identifying the educational content item in another interface or in another format. For example, the practitioner education module 235 transmits a notification message to a client device 150 of the medical practitioner that includes a link that, when selected by the medical practitioner, retrieves the selected educational content item for presentation.

The practitioner education module 235 may include a selected educational content item in one or more interfaces presented to the medical practitioner when accessing the collaborative medical platform 140 in various embodiments. For example, the practitioner education module 235 generates an interface including educational content and presents information describing a selected educational content item through the interface, allowing a medical practitioner to select the information describing the selected educational content item to access the selected educational content item. As another example, the practitioner education module 235 includes information identifying a selected educational content item in a medical case page generated by the interface management module 215 for a medical procedure for which the educational content item was selected. For example, a medical case page includes a section including notes or feedback for the medical practitioner about the medical case, with one or more educational content items selected by the practitioner education module 235 included in the section. Further, the interface management module 215 may generate one or more interfaces including recommendations for a medical practitioner based on metrics for the medical practitioner based on medical procedures, with the recommendation interface including one or more educational content items selected by the practitioner education module for the medical practitioner based on data describing performance of one or more medical procedures.

In some embodiments, the practitioner education module 235 includes a selected educational content item in different interfaces depending on content of the selected educational content item. For example, educational content items describing the use of a piece of medical equipment or of a medical instrument are displayed in a recommendation interface. As another example, educational content items comprising interactive material or audio or video data for presentation to a medical practitioner are presented in a medical case page or in an education interface. However, in other embodiments, the practitioner education module 235 selects an interface for identifying a selected educational content item based on other characteristics of the educational content item.

Alternatively or additionally, the practitioner education module 235 presents a selected educational content item to a medical practitioner during an intraprocedural stage of a medical procedure. This presents the selected educational content item to the medical practitioner while the medical practitioner performs the medical procedure. In various embodiments, the practitioner education module 235 transmits a notification identifying the selected educational content item to a piece of medical equipment 160 or to a client device 150 that displays the notification or audibly presents the notification to the medical practitioner. The notification may include specific content from the selected educational content item to simplify access to relevant information from the selected educational content item by the medical practitioner. In various embodiments, the practitioner education module 235 transmits a notification identifying an educational content item to a piece of medical equipment 160 associated with the educational content item. For example, the educational content item includes recommended settings for the piece of medical equipment 160 (e.g., force thresholds, movement thresholds), so transmitting the notification to the piece of medical equipment 160 simplifies identification of the medical equipment 160 relevant to the educational content item. A notification transmitted to a piece of medical equipment 160 may include a link that, when selected by the medical practitioner, causes the piece of medical equipment 160 to execute one or more instructions that modify one or more settings based on the educational content item. Similarly, information identifying an educational content item associated with a piece of medical equipment 160 presented by a client device 150 may include instructions that, when selected, transmit instructions for modifying one or more settings of the piece of medical equipment 160. This simplifies modification of settings of a piece of medical equipment 160 based on a selected educational content item by reducing an amount of interaction by the medical practitioner with the piece of medical equipment 160. Alternatively, the practitioner education module 235 includes information identifying a selected educational content item in an interface presented to the medical practitioner via a client device 150.

In some embodiments, a medical practitioner authorizes the practitioner education module 235 to automatically modify one or more settings of a piece of medical equipment 160 based on an educational content item selected for the medical practitioner. Such authorization may be specific to a particular medical procedure or limited to one or more specific pieces of medical equipment 160 used during a particular medical procedure. When the medical practitioner authorizes the practitioner education module 235 to automatically modify one or more settings of the piece of medical equipment 160, the practitioner education module 235 transmits a notification including one or more instructions corresponding to a selected educational content item to a piece of medical equipment 160 used in the medical procedure. The piece of medical equipment 160 executes the one or more instructions, modifying one or more settings of the piece of medical equipment 160 based on the selected educational content item. In various embodiments, the piece of medical equipment 160 displays a notification or otherwise notifies the medical practitioner that one or more settings have been modified or specified based on the selected educational content item. An indication that one or more settings are to be modified based on a selected educational content item may be presented to the medical practitioner by the piece of medical equipment 160 or by a client device 150 to alert the medical practitioner that one or more settings of the piece of medical equipment 160 are being automatically updated and provide the medical practitioner with an option to prevent modification of the one or more settings. Alternatively, the practitioner education module 235 automatically modifies one or more settings of a piece of medical equipment 160 based on an educational content item selected for a medical practitioner, as further described above, unless the medical practitioner indicates the practitioner education module 235 is not authorized to automatically modify one or more settings of a piece of medical equipment 160. This allows different embodiments to have a medical practitioner to opt-in to the practitioner education module 235 automatically modifying one or more settings of a piece of medical equipment 160 or to opt-out of the practitioner education module 235 automatically modifying one or more settings of a piece of medical equipment 160.

In other embodiments, presenting an educational content item during the intraprocedural stage of a medical case increases a number of interactions needed to modify one or more settings of a piece of medical equipment 160 used during a medical procedure. For example, presenting the educational content item via a piece of medical equipment 160 causes the piece of medical equipment 160 to request additional confirmation inputs from the medical practitioner subsequent to receiving input from the medical practitioner to change a specific setting of the piece of medical equipment 160 to a value deviating from a corresponding value int eh educational content item or to specify a particular value for the specific setting of the piece of medical equipment 160 outside of a range corresponding to the educational content item. As an example, presenting the educational content item to the medical practitioner transmits an instruction to a piece of medical equipment 160 used during the medical procedure that, when executed, causes the piece of medical equipment 160 to display one or more warnings each requesting an input from the medical practitioner when the piece of medical equipment 160 receiving an input from the medical practitioner to a value of a setting of the piece of medical equipment 160 to a value outside of a range included in the educational content item. This increases difficulty of the medical practitioner configuring the piece of medical equipment 160 in a manner that is inconsistent with the selected educational content item to increase a likelihood that values of settings of the piece of medical equipment 160 are consistent with the selected educational item.

The presentation module 240 leverages stored information associated with a completed medical procedure to facilitate generation of presentations for education, research, training, or other purposes. Presentations may be in the form of slide decks, posters, videos, animations, or other multimedia content. Presentations may incorporate various multimedia (e.g., video, images, three-dimensional models, and associated metadata), patient record data, medical equipment telemetry data, information from content feeds, analytics, or other information generated and/or stored by the collaborative medical platform 140.

In an embodiment, the presentation module 240 may maintain one or more presentation templates for generating presentations. The template may include pre-formatted content with various information fields that may be automatically populated from a set of records. For example, a practitioner wanting to prepare a presentation relating to a set of recently performed procedures may specify the set of procedures to include in the presentation, and the presentation module 240 may automatically populate the presentation based on the data stored in association with those procedures. pages, with each page associated with one or more types of data about the completed medical procedure. In some embodiments, the presentation module 240 may apply one or more trained machine learned models to automatically generate and/or recommend presentation content that may be of interest to a medical practitioner. In further embodiments, the presentation module 240 may intelligently automatically de-identify patient data included in the presentations.

The presentation module 240 may furthermore include various editing tools for creating, viewing, and editing presentations. For example, the editing tools may enable editing of text, video, images, animations, three-dimensional models, or other content for including in a presentation.

In an embodiment, presentations may be presented through a presentation module 240 directly without data associated with the presentation being exported externally to the collaborative medical platform 140. For example, the presentation module 240 may enable live streamlining of a presentation during a telepresence session to a set of invited attendees. The invited attendees may be limited to users 155 of the collaborative medical platform 140 or may include outside attendees that may gain access via an external link. Sharing presentations in this manner enables practitioners to maintain data privacy and compliance and avoid issues that may arise when externally exporting medical data.

The application integration module 245 manages integration of applications with the collaborative medical platform 140. Applications may be utilized to add additional optional functionality to the collaborative medical platform 140. For example, applications may enable integration with a specific EHR system, scheduling system, or other existing medical system. Applications may furthermore enable users to selectively add specific functionality beyond the core features of the collaborative medical platform 140. The application integration module 245 may allow third parties to create applications that interface with the collaborative medical platform 140 and make these applications available to add.

The application integration module 245 may maintain a catalog of applications capable of interfacing with the collaborative medical platform 140 and may provide interfaces to enable users 155 to selectively add applications for integration. In various embodiments, applications identified by the application integration module 245 have been authorized or approved for installation by an administrator of the collaborative medical platform 140, allowing regulation of the applications capable of executing on the collaborative medical platform 140.

Additionally, the application integration module 245 may include one or more application programming interfaces (API) for an application installed through the application integration module 245. An API for an application provides functionality for exchanging data between the application and one or more components of the collaborative medical platform 140, simplifying data exchange between the application and other portions of the collaborative medical platform 140.

The video library 250 stores videos of various medical procedures, training presentations, simulations, or other medical videos and metadata associated with the video. Examples of metadata associated with video of a medical procedure may include telemetry data of one or more medical instruments received in conjunction with the video, comments or annotations received from one or more medical practitioners through a surgical interface during the medical procedure included in the video, segmentation data that divides the video into temporal segments relating to different step of a procedure, profile information (e.g., age, body mass index, gender, etc.), associated with the patient in the video, or other information supplementing the video. Various reference content items including video data may be stored in the video library 250 for retrieval by the practitioner education module 235 in various embodiments.

The video library 250 may store videos in an indexed database that indexes videos based on various metadata. The video library 250 can then be browsed or searched via a video library interface to identify videos of relevance. The metadata associated with videos may include permissions stored in the connection graph store 255 that controls which users 155 have access to different videos. For example, a video in the video library 250 may be accessible only to users 155 that the video has been expressly shared with or that otherwise has viewing permissions for the video.

The connection graph store 255 comprises a database that stores information describing connections between entities or other objects (e.g., videos or other multimedia) managed by the collaborative medical platform 140. For example, as described above, the connection graph store 255 stores connections between users 155, connections between users 155 and procedures, connections between users 155 and multimedia content or other objects, or other connections between data entities of the collaborative medical platform 140.

The user profile store 260 stores profile data for users 155 of the collaborative medical platform 140. A user profile for a medical practitioner includes descriptive information such as a name of the medical practitioner, contact information for the medical practitioner, credentials or certifications of the medical practitioner, biographical information for the medical practitioner, types of medical procedures capable of being performed by the medical practitioner, medical facilities affiliated with the medical practitioner, operating room preferences (such as patient positioning, equipment setup, preferred instrumentation, typical procedure step order, etc.), equipment configuration preferences (e.g., ergonomic settings for a robot console), or other information describing the medical practitioner. Aspects of the user profile could be inferred using machine learning techniques. For example, a practitioner's preferred instrumentation or step order may be inferred from application of a machine learning model trained to infer such preferences based on observed historical data. Additionally, a user profile for a medical practitioner includes medical procedures performed by or to be performed by the medical practitioner, as well as information describing the medical procedures. For example, the user profile identifies different types of medical procedures to be performed by, or performed by, the medical practitioner, and may include characteristics for each medical procedure (e.g., a length of time to complete the medical procedure, a number of times the medical practitioner performed a type of medical procedure matching the medical procedure, etc.). Further, one or more of the metrics determined by the analytics module 230 for the medical practitioner, as further described above, may be included in the user profile for the medical practitioner.

The patient data store 265 includes a patient profile for each patient associated with medical cases. A patient profile includes characteristics of a corresponding patient, which may be obtained from an electronic health record for the patient or may be provided via input from a medical practitioner. Characteristics of a patient include demographic information about the patient, medical conditions of the patient, medical procedures previously performed by the patient, allergies of the patient, contact information for the patient, current or prior prescriptions for the patient or other medically relevant information about the patient. A patient identifier is associated with a patient profile to uniquely identify the patient profile.

All of the data stored to the collaborative medical platform 140 (or otherwise made available through the collaborative medical platform 140) may be stored, presented, and in some cases restricted in a manner that ensures compliance with various data privacy and protection regulations.

FIGS. 3A-3B illustrate an example practitioner dashboard 300. FIG. 3A shows an upper portion of the dashboard 300 while FIG. 3B shows a lower portion of the dashboard 300 (which may be continuously scrollable). The practitioner dashboard 300 may operate as a home landing page for a medical practitioner upon logging into the collaborative medical platform 140. The practitioner dashboard 300 may include various content sections, at least some of which may be specifically targeted to the practitioner. A search bar 305 enables input of text-based search queries for searching content available in the collaborative medical platform 140 (e.g., case pages, other user pages, videos, presentations, etc.). In response to inputting a search query, a list of results may be displayed with links to content matching the search query. A video promotion section 310 shows a video recently added by the practitioner with user interface tools to enable the practitioner to promote the video by sharing it with other users, create a highlight reel, or view various statistical information about the video. An achievement section 315 presents an achievement relating to use of the collaborative medical platform 140. In this example, the achievement section 315 highlights that the user has recently reached 100 videos and provides links to view the user's videos and access a video library. Other examples of achievements in the achievement section 315 could relate to number of cases managed, time using the platform 140, number of connections, count of frequency of interactions, or other usage achievements. The video library 320 includes video thumbnails, video tags, or other links to enable browsing of videos selected as potentially relevant to the medical practitioner. For example, relevant videos may be selected that relate to past or upcoming procedures associated with the medical practitioner, based on a history of videos viewed by the medical practitioner, based on a practice area or other profile information for the medical practitioner, or other factors. The webinar promotion section 325 includes a promotional banner for an upcoming webinar that will be viewable within the collaborative medical platform 140. The webinar may be identified as being of potential interest to the medical practitioner based on, for example, the subject matter of the webinar, the host of the webinar, or other factors. The shared cases section 330 provides summary information and links to case pages that have been shared with the medical practitioner. Examples of case pages are described in further detail below. The analytics summary 335 includes example analytics associated with the medical practitioner's usage of the collaborative medical platform 140, procedures performed by the medical practitioner, or other analytics data derived from information stored in the collaborative medical platform 140. The analytical data may be presented in one or more visual representations such as a graph or chart. The feedback section 340 provides links to enable the medical practitioner to send feedback to an administrator of the collaborative medical platform 140.

FIGS. 3A-3B illustrate just one example of a practitioner dashboard 300. The types of content presented in the practitioner dashboard 300 may be different for different practitioners and/or may dynamically change over time for the same medical practitioner. Some of the sections may be fixed and always appear upon accessing the dashboard 300 (e.g., the search bar 305, video library 320, shared cases 330, analytics 335, and feedback sections 340), while other sections (e.g., video promotion 310, achievement 315, webinar promotion 325) may be dynamically inserted only in certain contexts. For example, webinar promotions 325 may be presented only when an upcoming webinar deemed to be of sufficient interest is upcoming. Achievements 315 may similarly be displayed only when a relevant achievement has recently been achieved. Furthermore, the dashboard 300 could be customized by the user to display desired sections in a configured order. The various sections 305, 310, 315, 320, 325, 330, 335, 340 when present, may furthermore be presented in different order in different contexts.

FIG. 4 shows an alternative embodiment of a practitioner dashboard 400. In the example shown by FIG. 4, the practitioner dashboard 400 includes an educational content item section 405 including information identifying an educational content item selected for a medical practitioner based on a previously performed medical procedure. The practitioner education module 235 selects the identified educational content item based on stored baseline criteria for educational content items associated with a type of the previously performed medical procedure and captured data describing performance of the previously performed medical procedure, as further described above in conjunction with FIG. 2. In various embodiments, the educational content item section 405 identifies one or more reasons why the identified educational content item is of potential interest to the medical practitioner. In the example of FIG. 4, the educational content item section 405 indicates that the identified educational content item includes suggested parameters or settings for a piece of medical equipment 160 (e.g., a robotic arm) used in the previously performed medical procedure. The educational content item section 405 in the example of FIG. 4 includes a link 410 that, when selected by the medical practitioner retrieves the educational content item for presentation to the medical practitioner via a client device 150 of the medical practitioner.

For purposes of illustration, FIG. 4 shows an example practitioner dashboard 400 where the educational content item section 405 is displayed proximate to a search bar 305. For example, the educational content item section 405 is displayed in a position of the practitioner dashboard 400 below the search bar 305, so the educational content item section 405 is prominently displayed in the practitioner dashboard 400 to increase a likelihood of the medical practitioner selecting the link 410 to the identified educational content item. However, in other embodiments, the practitioner dashboard 400 displays the educational content item section 405 in a different position relative to other sections. Similarly, while FIG. 4 shows an example where the achievement section 315 and the video library section 320 are displayed in conjunction with the educational content item section 405, in other embodiments, different or additional sections are displayed by the practitioner dashboard 400 in conjunction with the suggested reference content item section 405.

In various embodiments, the educational content item section 405 is dynamically inserted into the practitioner dashboard 400 in certain contexts and is not included in the practitioner dashboard 400 in other contexts. For example, the practitioner dashboard 400 displays the educational content item section 405 after the medical practitioner has completed a medical procedure. In an example, the practitioner dashboard 400 displays the educational content item section 405 starting a specific amount of time after the medical practitioner completed a medical procedure, but does not display the educational content section 405 before the specific amount of time lapses after completion of the medical procedure. The practitioner dashboard 400 displays the educational content item section 405 for a particular time interval after the medical practitioner completed the medical procedure in various embodiments.

While FIG. 4 shows an example where the educational content item section 405 identifies a single reference content item, in other embodiments, the educational content item section 405 displays multiple reference content items selected for the medical practitioner. For example, the educational content item section 405 is a carousel content item having multiple slides, with each slide including information identifying a different selected educational content item and including a link to a different selected educational content item. In response to the medical practitioner performing a specific interaction with the educational content item section 405, the educational content item section 405 is updated to display a different slide including information identifying a different selected educational content item. For example, the educational item section 405 displays an alternative slide including information identifying a different selected educational content item in response to the medical practitioner performing a swiping gesture along an axis perpendicular to an axis including the search bar 305, the educational content item section 405, the achievement section 315, and the video library section 320. This allows a single section of the practitioner dashboard 400 to identify multiple educational content items to the medical practitioner.

In some embodiments, the collaborative medical platform 140 generates one or more education interfaces, such as an education dashboard. An education interface may additionally or alternatively display one or more suggested educational content items to a medical practitioner, providing an additional way for the medical practitioner to access the suggested educational content items. The practitioner dashboard 400 may include an interface element that, when selected by the medical practitioner, causes display of the education interface. The education interface may display information identifying multiple suggested educational content items in some embodiments, allowing the medical practitioner to more easily access a wider range of suggested educational content items.

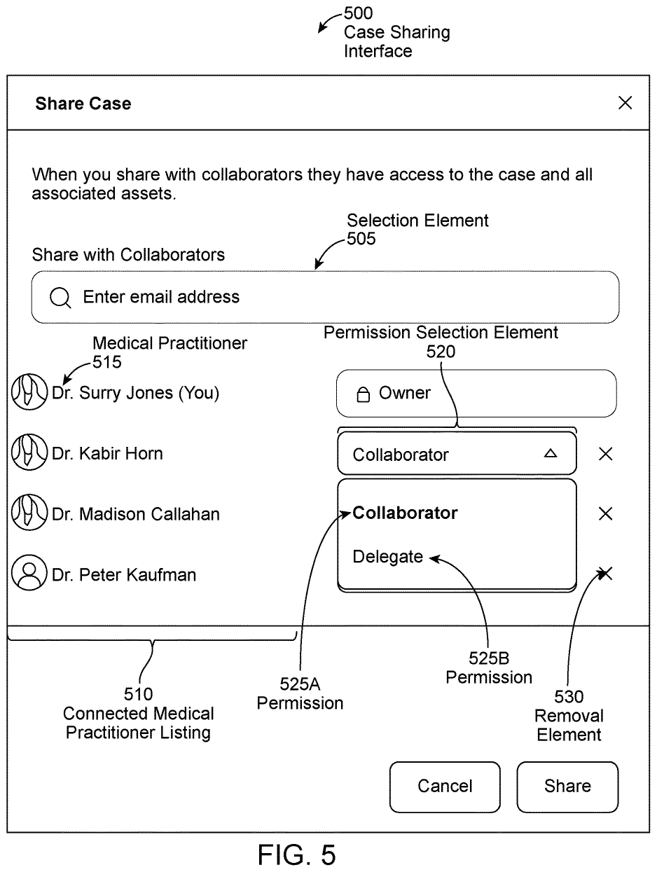

FIG. 5 shows an example embodiment of a case sharing interface 500 for sharing a case with one or more contributors. Adding a contributor to a case may generate a connection between the contributor and the case and between the contributor and the case owner. The case sharing interface 500 includes a selection element 505 for receiving identifying information to identify a desired contributor. For example, the selection element 505 may receive an email address, name, a username, or another identifier of a medical practitioner or other requested contributor. In some embodiments, upon selecting identifying information for a desired contributor, the case sharing interface 500 may display all or a portion of profile data for the requested collaborator to enable the requestor to confirm if the matched profile data is the intended collaborator. The case sharing interface 500 then enables the requestor to confirm or decline selection of a collaborator and interact with a permission selection element 520 to set a desired permission level for the requested collaborator. Here, the permission level may place limits on an invited collaborator's access to data about the case and/or may limit actions the collaborator is permitted to perform in association with the case. In an example embodiment, the permission level may be selected between a “collaborator” level 525A and a “delegate” level 525B.

In response to receiving inputs to select a requested collaborator and set a desired permission level (via the permission selection element 520), the case sharing interface 500 may send an invitation to the requested contributor (e.g., via an email, text message, phone call, portal message, or other communication mechanism) to enable the requested collaborator to accept or decline the request. If the request is accepted, the case sharing interface 500 may add the identifier or other information for the new collaborator to a connected medical practitioner listing 510 that lists the contributors added to the case. For example, the illustrated example shows a connected medical practitioner listing 510 that includes the case owner 515 and three additional contributors that have been added to the case.

The case sharing interface 500 may furthermore enable the case owner to change permission levels of existing contributors in the connected medical practitioner listing 510. Furthermore, the case sharing interface 500 may include removal elements 530 associated with each contributor in the connected medical practitioner listing 510 that enables removal of a contributor from the case. Selection of a removal element 530 may remove the stored connection in between the practitioner and the case, such that the practitioner no longer has access to the case.

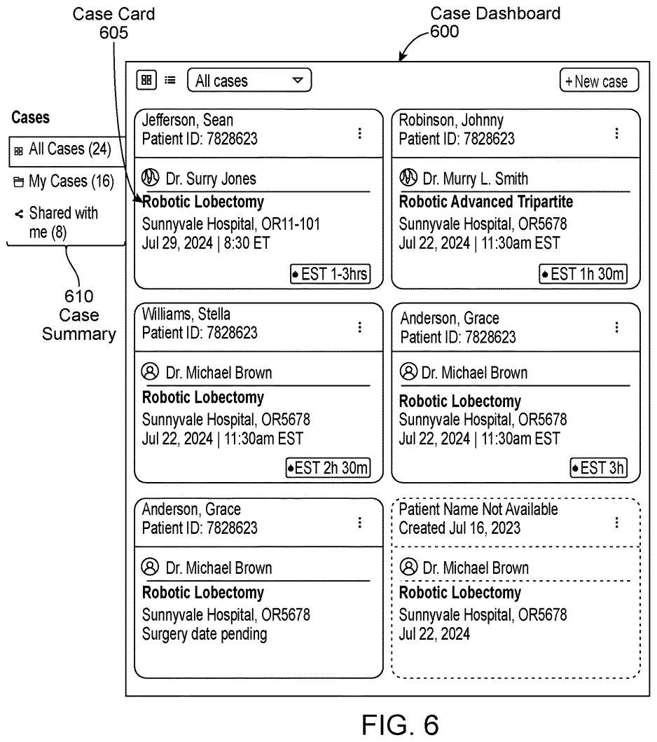

FIG. 6 is an example embodiment of a case dashboard 600 for a medical practitioner. The case dashboard 600 enables access to cases owned by the medical practitioner and cases shared with the medical practitioner by other users 155 as indicated in the case summary 610. In this example, the case dashboard 600 is organized as a set of case cards 605 that each graphically show a summary of a case. Selecting a case card 605 links to a case page 400 for the case. In alternative embodiments, the dashboard 600 may be presented in a list view or other view without necessarily presenting case cards 605 in the visual form shown in FIG. 6.

FIG. 7 shows an example of a telepresence interface 700 associated with a telepresence session that may take place during an actual procedure or during a simulated procedure. Alternatively, the telepresence session may be utilized for live planning purposes without necessarily performing or simulating a procedure. In this example, the telepresence interface 700 displays a three-dimensional model of a target anatomy 705 associated with the procedure. The model may include annotated comments that may be obtained during the telepresence session or that were added in a preprocedural stage. Alternatively, the telepresence interface 700 may include a view of real-time video or images associated with an ongoing procedure. In an embodiment, each contributor may be able to switch between different relevant views such as real-time video or images, three-dimensional models, preprocedural images, or other relevant multimedia.

The telepresence interface 700 may furthermore include a telepresence content feed 715 for sending and receiving real-time messages between contributors. For example, a telepresence content feed 715 allows users to post messages and/or view messages from other participants. The messages may include text, media content (e.g., images, video, animations, etc.), or links to various media content or other resources (e.g., research articles). The telepresence interface 700 may furthermore enable participants to provide annotations on the target anatomy (presented in the form of an image, video, or model). For example, a participant may pin a comment to a specific location in the depicted anatomy, as may be indicated by an identifier 710.

Additionally, the telepresence interface 700 may display statistics 720 or other analytics that may be relevant to the procedure. The statistics 720 maybe include estimated or modeled values or metrics relating to the anatomy based on various sensed data from the medical equipment 160. The telepresence interface 700 may dynamically update the statistics 720 over time during the procedure.

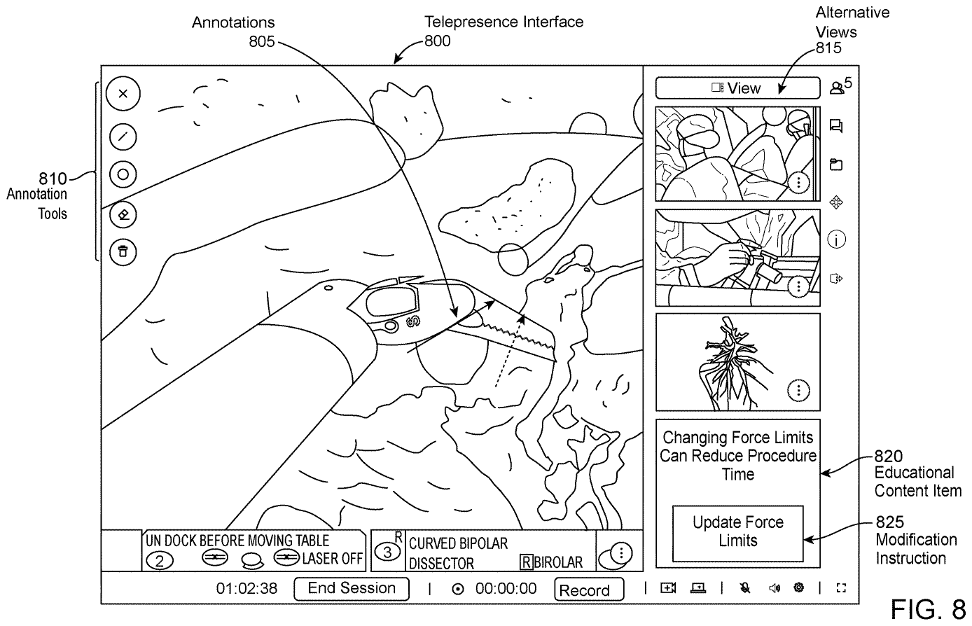

FIG. 8 is another example of a telepresence interface 800 associated with a telepresence session. In this example, the telepresence interface 800 shows a live video of a procedure being performed together with a set of annotation tools 810 that enables a remote contributor to add annotation 805 overlaid on the video. The telepresence interface 800 also includes a set of alternative views 815 the contributor can switch between during the telepresence session. These alternative views 815 may include one or more different camera views (e.g., a view of the medical environment), one or more three-dimensional models (e.g., as shown in FIG. 7), views of preprocedural images, or other multimedia associated with the case. In various embodiments, telepresence interfaces, such as shown in FIG. 7 or 8, are stored for a medical case and may be subsequently presented to other medical practitioners if a medical practitioner associated with the case authorizes generation of a reference case based on the medical case, as further described above in conjunction with FIG. 2.

In the example of FIG. 8, the telepresence interface 800 also displays an educational content item 820 to a medical practitioner, such as the medical practitioner performing the medical procedure. The educational content item 820 is dynamically selected by the telepresence interface 800 in various embodiments based on captured telemetry data or video data during the medical procedure. The telepresence interface 800 includes information describing the educational content item 820 or extracted from the educational content item 820, allowing the medical practitioner to discern content from the educational content item 820 via the telepresence interface 800. In various embodiments, the telepresence interface 800 limits presentation of the educational content item 820 to certain time intervals. For example, the telepresence interface 800 displays the educational content item 820 in response to the collaborative medical platform 140 determining that telemetry data or video data captured during performance of the medical procedure deviates by at least a threshold amount from baseline criteria associated with the educational content item 820. When captured telemetry data or video data does not deviate by at least the threshold amount from the corresponding baseline criteria for the educational content item 820, the telepresence interface 800 does not present the educational content item 820. For example, a client device 150 displaying the telepresence interface 800 receives a presentation instruction to present the educational content item 820 along with the educational content item 820 from the collaborative medical platform 140 and subsequently receives an alternative instruction to stop presenting the educational content item 820 from the collaborative medical platform 140. The alternative instruction may be received in response to the collaborative medical platform 140 determining telemetry data or video data received during performance of the medical procedure satisfies baseline criteria associated with the educational content item 820 or in response to determining telemetry data or video data no longer identifies a pattern corresponding to a baseline criterion associated with the educational content item 820.

To simplify incorporation of information from the educational content item 820 into the medical procedure, the telepresence interface 800 presents a modification instruction 825 in association with the educational content item 820. The modification instruction 825 includes an identifier of a piece of medical equipment 160 and values of one or more settings for the piece of medical equipment 160. In response to the medical practitioner selecting the modification instruction 825 via the telepresence interface 800, the collaborative medical platform 140 receives a request identifying the educational content item 820 and the piece of medical equipment 160. In response to receiving the request, the collaborative medical platform 140 transmits an instruction to the identified piece of medical equipment 160 to modify values of one or more settings to values retrieved from the educational content item 820 and included in the instruction transmitted to the piece of medical equipment 160. In various embodiments, the collaborative medical platform 140 determines the identifier of the piece of medical equipment 160 based on an identifier included in telemetry data received by the collaborative medical platform 140 or based on identifying information included in received video data of the medical procedure. This simplifies modification of one or more settings of the piece of medical equipment based on the educational content item 820 via interaction with the telepresence interface 800 rather than by manually entering values for settings identified by the educational content item to the piece of medical equipment 160.

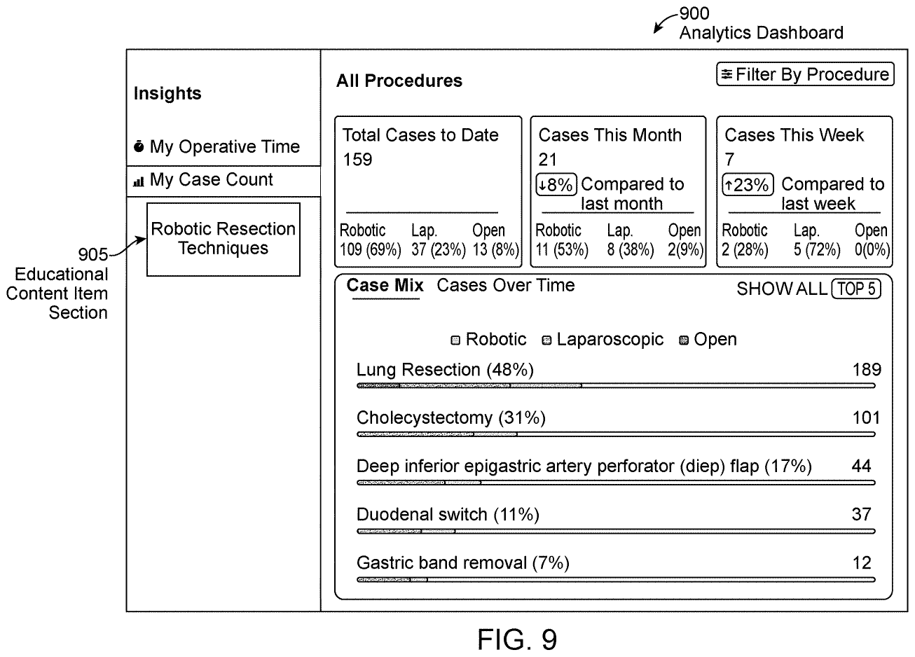

FIG. 9 is an example embodiment of an analytics dashboard 900 for a medical practitioner. In this example, the analytics dashboard 900 displays a summary of cases managed by the medical practitioner includes, for example, a total number of cases, a number of cases in the current month, a number of cases in the current week, and a distribution of types of cases the practitioner has performed. In the example of FIG. 9, the analytics dashboard 900 also displays an educational content item section 905 to the medical practitioner. The educational content item section 905 includes information identifying an educational content item the collaborative medical platform 140 selected for the medical practitioner based on data describing performance of a medical procedure by the medical practitioner, as further described above in conjunction with FIG. 2. The educational content item section 905 includes a link that, when accessed, retrieves the educational content item from the collaborative medical platform 140 or from a third-party server 170 for presentation in various embodiments. The educational content item section 905 may identify an educational content item selected based on a medical procedure most recently completed by the medical practitioner in some embodiments. Alternatively, the analytics dashboard 900 includes multiple educational content item sections 905, with each educational content item section including an educational content item selected for a medical procedure previously performed by the medical practitioner, simplifying access to different educational content items relevant to various medical procedures performed by the medical practitioner.

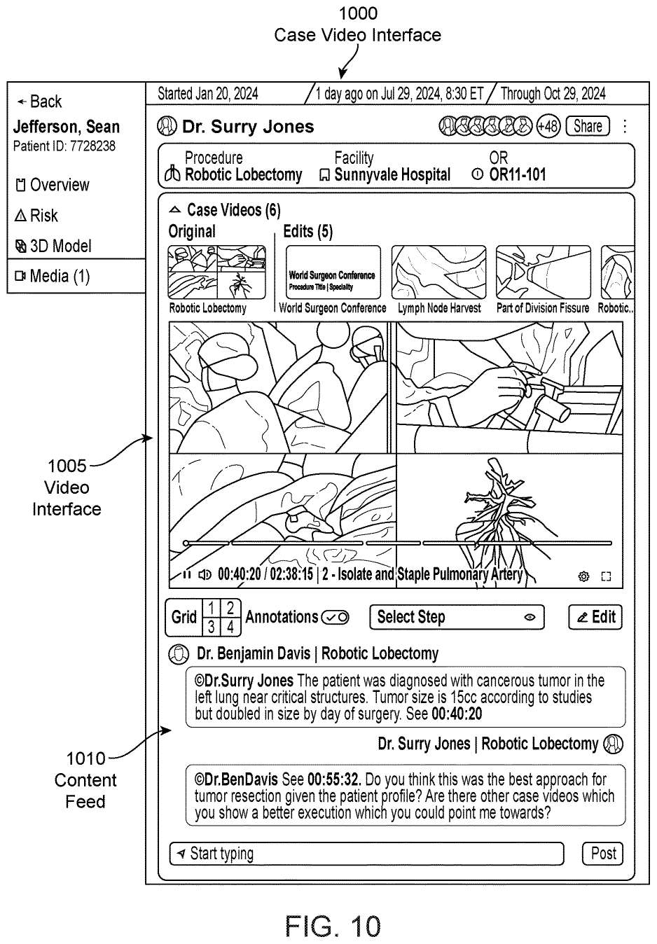

FIG. 10 is an example embodiment of case video interface dashboard 1000 for viewing a case video. Case videos may be captured during a telepresence session or may be similarly captured during a procedure without a live streamed telepresence session. The case video interface 1000 includes a video interface 1005 that shows one or more views of a video associated with a medical procedure. The video interface 1005 may include multiple captured views, which may be from cameras in the medical environment, cameras inserted into the anatomy (e.g., endoscopy cameras), or other cameras. Captured views may furthermore include three-dimensional models, preprocedural images, procedure planning documents, or other visual information. The video may be segmented (manually or automatically using video processing and content recognition techniques) to divide the video into segments associated with different steps of the procedure. The video may include annotations provided by a medical practitioner during a telepresence session or in a postprocedural review. A content feed 1010 may be presented in association with a video to enable users 155 to post comments, links, media, or other content in association with the presentation. A reference content item, such as a reference case, may display video and other information (e.g., a content feed 1010) of a medical procedure to a medical practitioner using the video interface 1000 described in conjunction with FIG. 10.

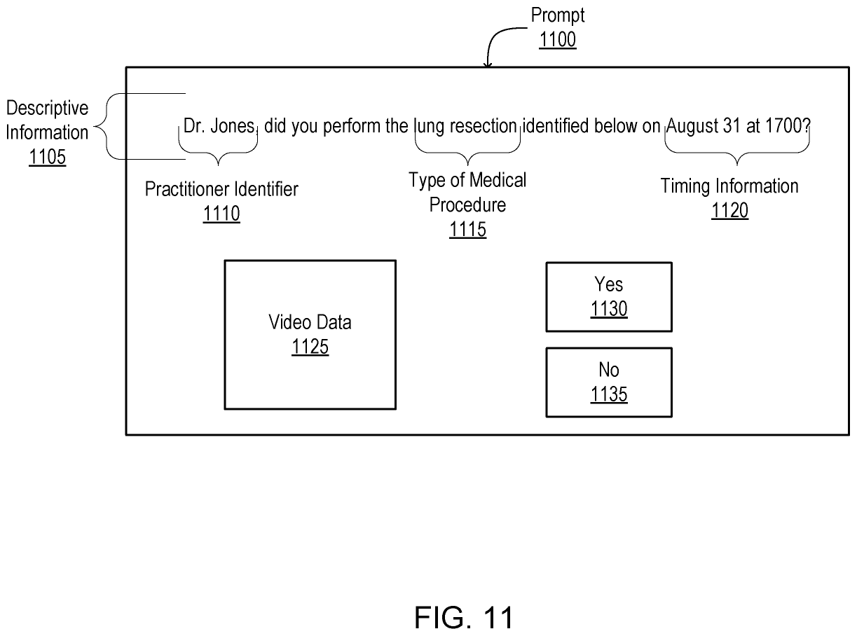

FIG. 11 is an example prompt for a medical practitioner to confirm a connection to telemetry data or video data captured during a medical procedure. The prompt 1100 is presented to a medical practitioner selected by the analytics module 230 based on the telemetry data or video data. In various embodiments, the prompt 1100 is presented to the medical practitioner via a client device 150 after the analytics module 230 selects the medical practitioner, as further described above in conjunction with FIG. 2. For example, the collaborative medical platform 140 transmits the prompt 1100 to a client device 150 from which information identifying the medical practitioner was received after the analytics module 230 selected the medical practitioner. As an example, the prompt 1100 is presented as an overlay on a portion of the practitioner dashboard 300, further described above in conjunction with FIGS. 3A and 3B. Alternatively, the prompt 1100 is presented as a portion of the practitioner dashboard 300 presented to the medical practitioner when the medical practitioner accesses the collaborative medical platform 140.

In other embodiments, the collaborative medical platform 140 transmits the prompt 1100 using alternative communication channels or alternative formats. For example, the prompt 1100 comprises a push notification transmitted from the collaborative medical platform 140 to a client device 150 of the medical practitioner. In another example, the prompt is included in an email or in a text message transmitted from the collaborative medical platform 140 to a client device 150 of the medical practitioner. Further, in some embodiments, the collaborative medical platform 140 transmits the prompt 1100 to the medical practitioner using multiple communication channels or multiple formats. For example, the collaborative medical platform 140 transmits the prompt 1100 as a push notification to the client device 150 of the medical practitioner and displays as an overlay on a portion of the practitioner dashboard 300, as shown in FIG. 11.

The prompt 1100 includes descriptive information 1105 of telemetry data or video data received by the collaborative medical platform 140. As further described above in conjunction with FIG. 2, in various embodiments, the descriptive information 1105 includes metadata extracted from the telemetry data or the video data. In the example shown by FIG. 11, the descriptive information 1105 also includes a practitioner identifier 1110 of the medical practitioner to whom the prompt 1100 is presented. Example metadata determined from the telemetry data or video data includes a type 1115 of the medical procedure during which the telemetry data or video data was captured.

In some embodiments, the collaborative medical platform 140 includes additional example metadata extracted from the telemetry data or video data includes timing information 1120 indicating when the telemetry data or video data was captured. However, different or additional types of metadata may be included in the descriptive information 1105 for the telemetry data or the video data. For example, the prompt includes at least a portion of the video data 1125, as shown in the example the example shown in FIG. 11. The prompt 1100 may alternatively or additionally include at least a portion of the telemetry data in various embodiments. However, in other embodiments, the prompt 1100 does not include descriptive information 1105 of the telemetry data or video data and does not include metadata extracted from the telemetry data or video data, with the prompt 1100

The prompt 1100 also includes a confirmation interface element 1130 and a rejection interface element 1135. In response to receiving a selection of the confirmation interface element 1130 from the medical practitioner, the client device 150 presenting the prompt 1100 transmits a confirmation that the telemetry data or video data is associated with the selected medical practitioner to the collaborative medical platform 140. Subsequently, the collaborative medical platform 140 stores a connection between the medical practitioner and the telemetry data or video data. However, in response to receiving a selection of the rejection interface element 1135, the client device 150 presenting the prompt 1100 transmits a rejection to the collaborative medical platform 140, so the collaborative medical platform 140 does not store a connection between the medical practitioner and the telemetry data or video data. In some embodiments, the collaborative medical platform 140 selects an alternative medical practitioner, as further described above in conjunction with FIG. 2, in response to receiving the rejection. The collaborative medical platform 140 subsequently modifies the prompt 1100 and presents the modified prompt 1100 to the alternative medical practitioner. Alternatively, the confirmation interface element 1130 and the rejection interface element 1135 are presented to the medical practitioner in response to the collaborative medical platform 140 receiving a selection of the prompt 1100, rather than being included in the prompt 1100.

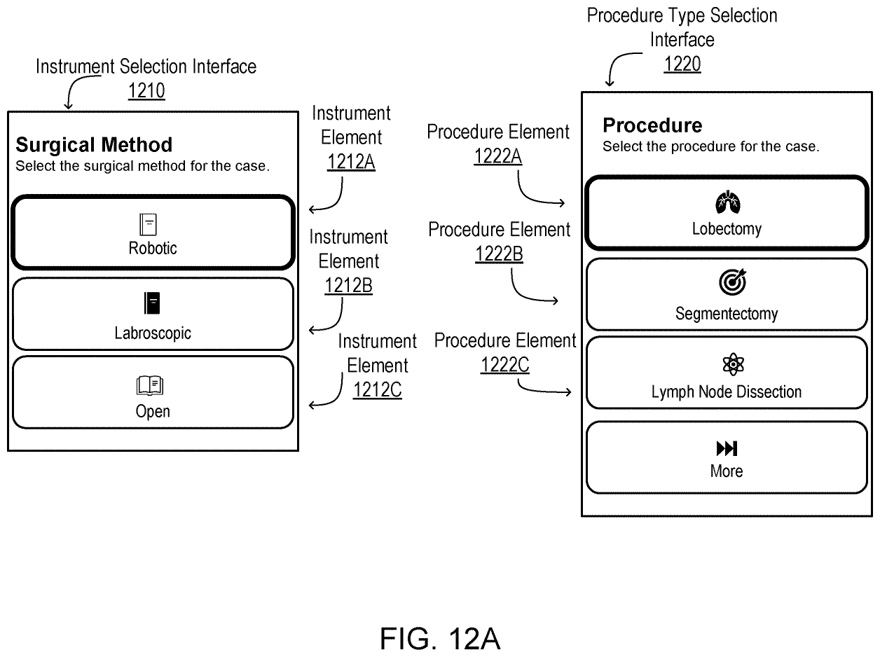

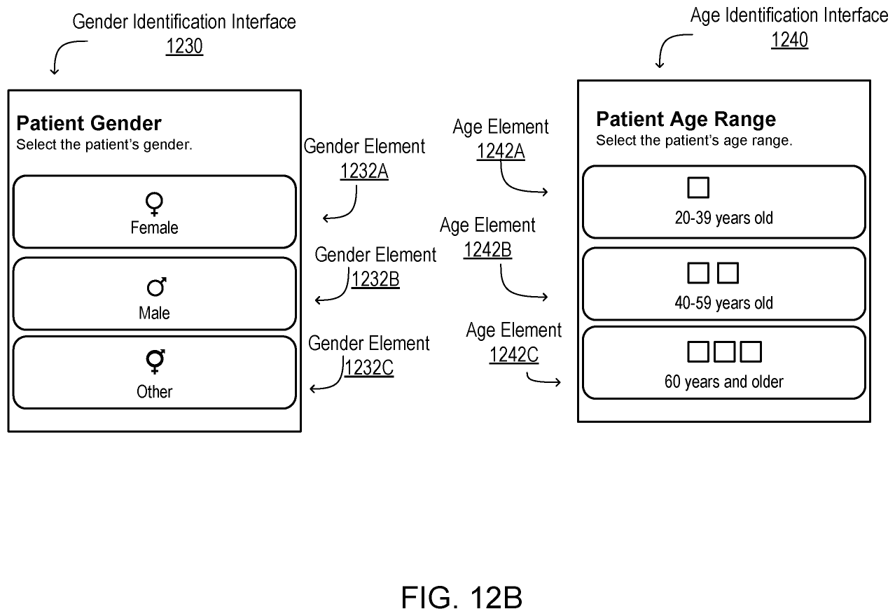

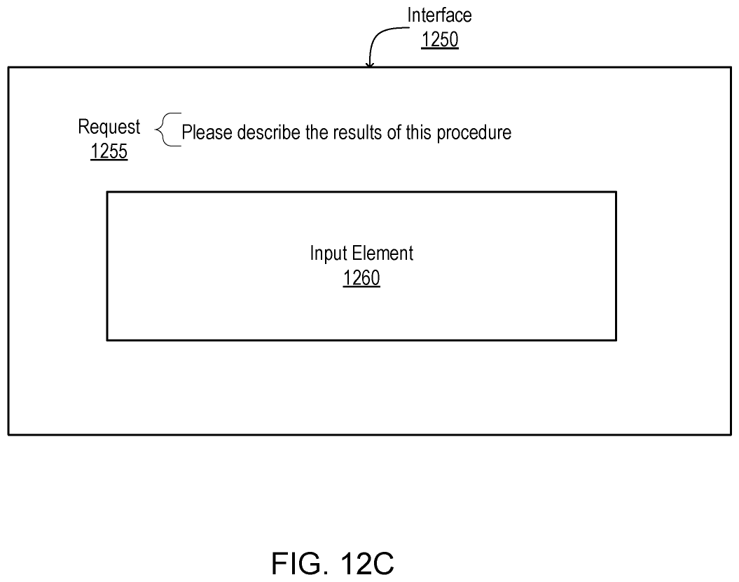

FIGS. 12A-12C are example interfaces requesting supplemental information for telemetry data or video data connected to a medical practitioner. In various embodiments, one or more of the example interfaces are presented to the medical practitioner in response to receiving a specific interaction with the prompt 1100 from the medical practitioner. For example, one or more of the example interfaces described in conjunction with FIGS. 12A-12C are presented to the medical practitioner via a client device 150 in response to the collaborative medical platform receiving a selection of the confirmation interface element 130.

For purposes of illustration, FIGS. 12A-12B show examples of an instrument selection interface 1210, a procedure type selection interface 1220, a gender identification interface 1230, an age identification interface 1240, and a procedure description interface 1250. However, in other embodiments, different or additional interfaces are presented by the collaborative medical platform 140. Further, in some embodiments, the collaborative medical platform 140 combines one or more of the interfaces described in conjunction with FIGS. 12A-12C into a single interface.

The instrument selection interface 1210 shown in FIG. 12A includes multiple instrument elements 1212A-1212B (also referred to individually or collectively using reference number 1212). Each instrument element 1212 identifies a different type of medical instrument, or piece of medical equipment 160 used in a medical procedure. For example, instrument element 1212A identifies a robot, instrument element 1212B identifies a laparoscope, and instrument element 1212C identifies the medical procedure was performed without one or more specific types of instruments (e.g., pieces of medical equipment 160, medical instruments). The medical practitioner selects one or more of instrument element 1212A, 1212B, 1212C, and the collaborative medical platform 140 stores an identifier of the selected one or more instrument elements 1212A, 1212B, 1212C in association with the medical procedure. Hence, the instrument selection interface 1210 reduces an amount of input from the user to identify one or more types of medical instrument (or pieces of medical equipment 160) associated with the telemetry data or video data.

The procedure type selection interface 1220 shown in FIG. 12A includes multiple procedure elements 1222A-1222B (also referred to individually or collectively using reference number 1222). Each procedure element 1222 identifies a different type of medical procedure. For example, each of procedure element 1222A, procedure element 1222B, and procedure element 122C identify a different type of medical procedure. Further, the procedure type selection interface 1220 may include additional interface elements for retrieving additional type of medical procedure or for receiving input from a medical practitioner identifying a type of medical procedure. The collaborative medical platform 140 may apply one or more classification models to the telemetry data or video data and select a subset of types of medical procedures corresponding to different procedure elements 1222 in some embodiments. Application of the configuration model would allow the procedure type selection interface 1220 to dynamically modify the types of medical procedure initially presented by procedure elements 1222 in the procedure type selection interface 1220 based on characteristics or attributes of the telemetry data or video data. In various embodiments, the medical practitioner selects one of procedure element 1222A, 1222B, 1222C, and the collaborative medical platform 140 stores an identifier of the type of medical procedure corresponding to the selected procedure element 1222 in association with the medical procedure. Hence, the instrument selection interface 1220 reduces an amount of interaction by the medical practitioner with the collaborative medical platform 140 to identify a type of medical procedure during which the telemetry data or video data was captured.

While the instrument selection interface 1210 and the procedure type selection interface 1220 obtain information from the medical practitioner about the medical procedure or instruments used during the medical procedure during which the telemetry data or video data was captured, the collaborative medical platform 140 also obtains information from the medical practitioner about the patient on whom the medical procedure was performed. The gender identification interface 1230 and the age identification interface 1240 shown in FIG. 12B receive input from the medical practitioner identifying characteristics of the patient on whom the medical procedure was performed. The gender identification interface 1230 includes multiple gender elements 1232A-C (also referred to individually and collectively using reference number 1232). Each gender element 1232A-C corresponds to a different gender of the patient, and the collaborative medical platform 140 stores a gender corresponding to a gender element 1232 selected by the medical practitioner in association with the telemetry data or video data.

Similarly, the age identification interface 1240 includes multiple gender elements 1242A-C (also referred to individually and collectively using reference number 1242). Each age element 1242A-C corresponds to a different range of ages for the patient. The collaborative medical platform 140 stores an age range corresponding to an age element 1242 selected by the medical practitioner in association with the telemetry data or video data. In other embodiments, different interfaces may request additional or alternative information about a patient on which the medical procedure was performed.

Additionally, the collaborative medical platform 140 may present the procedure description interface 1250 shown in FIG. 12C to the medical practitioner. In the example of FIG. 12C, the procedure description interface 1250 presents a request 1255 determined by the collaborative medical platform 140 to a medical practitioner. In various embodiments, the collaborative medical platform 140 presents the procedure description interface 1250 to a medical practitioner in response to receiving a confirmation from the medical practitioner that telemetry data or video data received by the collaborative medical platform 140 is connected to the medical practitioner. The procedure description interface 1250 may be presented after the interfaces further described above in conjunction with FIGS. 12A and 12B were presented to the medical practitioner in some embodiments. The procedure description interface 1250 allows the collaborative medical platform 140 to receive supplemental information augmenting the video data or the telemetry data from the medical practitioner connected to the video data or the telemetry data.

The request 1255 identifies or describes supplemental information to receive from the medical practitioner about telemetry data or video data connected to the medical practitioner by the collaborative medical platform 140. In various embodiments, the collaborative medical platform 140 generates the request 1255 by applying a generative model, such as an LLM, to the telemetry data or video data and to characteristics of the medical practitioner. Alternatively, the collaborative medical platform 140 stores predefined requests from which the request 1255 is selected and presented.

To receive supplemental information based on the request 1255, the procedure description interface 1250 includes an input element 1260, such as a text box, that receives input from the medical practitioner related to the request 1255. The collaborative medical platform 140 stores data received from the medical practitioner via the input element 1260 as supplemental information associated with the telemetry data or video data. In some embodiments, the procedure description interface 1250 also includes an interface element that, when selected by the medical practitioner, transmits the data received via the input element 1260 to the collaborative medical platform 140 in conjunction with an identifier of the medical practitioner and an identifier of telemetry data or video data.

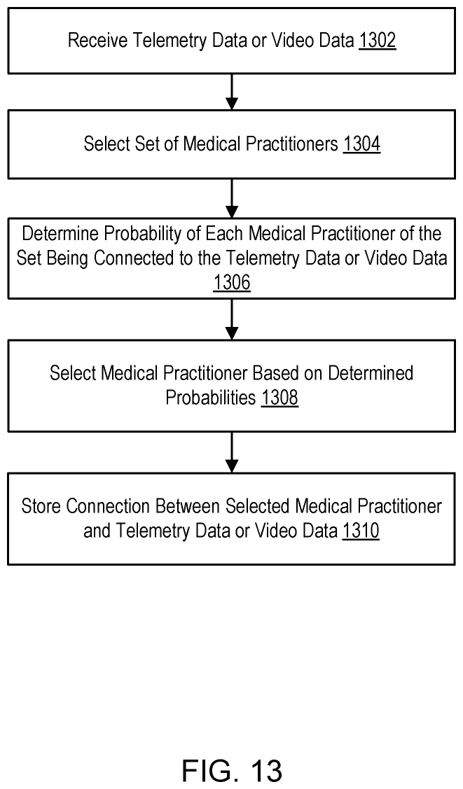

FIG. 13 is an example embodiment of a process for a collaborative medical platform 140 selecting a medical practitioner connected to received telemetry data or video data. The collaborative medical platform 140 receives 1302 telemetry data or video data from a location, such as a medical facility. The telemetry data or video data was captured during performance of a medical procedure at the location. As further described above in conjunction with FIG. 2, the telemetry data includes describing values of settings for one or more pieces of medical equipment 160 used during the medical procedure, configuration data for one or more pieces of medical equipment 160, or other information describing operation or functioning of one or more pieces of medical equipment 160. The video data includes portions of one or more medical practitioners associated with the medical procedure, portions of one or more pieces of medical equipment 160 (or medical instruments) during the medical procedure, portions of a patient on whom the medical procedure was performed, or other portions of the location where the medical procedure was performed.

However, the telemetry data or video data does not include information identifying the medical procedure during which it was captured or one or more medical practitioners associated with the medical procedure during which the telemetry data or video data was captured. Various locations, such as medical facilities, are subject to data privacy restrictions that prevent transmission of information capable of uniquely identifying a patient on which a medical practitioner was performed to systems external to the location. For example, a hospital may transmit the telemetry data or video data to the collaborative medical platform 140, but is prevented from providing information identifying the medical procedure during which the telemetry data or video data was captured or identifying one or more medical practitioners associated with the medical procedure to the collaborative medical platform. Similarly, the collaborative medical platform 140 may be prevented from accessing scheduling information for one or more medical practitioners at a location, such as a medical facility, as such access could allow the collaborative medical platform 140 to infer a patient on whom a medical practitioner performed a medical procedure.

Receiving 1302 telemetry data or video data without information identifying a medical practitioner prevents the collaborative medical platform 140 from providing a medical practitioner connected to the telemetry data or video data with information generated from the telemetry data or video data. For example, one or more metrics describing performance of a medical procedure during which the telemetry data or video data was captured are unable to be provided to a medical practitioner performing the medical procedure if the collaborative medical platform 140 is unable to determine a medical practitioner connected to the telemetry data or video data. Similarly, educational content the collaborative medical platform 140 may determine for a medical practitioner based on the telemetry data or video data is unable to be provided to a medical practitioner associated with the medical procedure during which the telemetry data or video data was captured. As locations often perform multiple medical procedures during a time interval, the collaborative medical platform 140 may receive 1302 a large amount of telemetry data or video data from the location, making it impractical to review telemetry data or video data and information about medical practitioners maintained by the collaborative medical platform 140 to identify connections between a medical practitioner and received telemetry data or video data.

Hence, to identify a medical practitioner associated with the telemetry data or video data, the collaborative medical platform 140 selects 1304 a set of medical practitioners based on the telemetry data or video data. In various embodiments, each medical practitioner of the set has a common characteristic. For example, each medical practitioner of the set is associated with a location matching the location from which the telemetry data or video data was received 1302 by the collaborative medical platform 140. As an example, each medical practitioner of the set has a user profile maintained by the collaborative medical platform 140 identifying a medical facility matching a medical facility identified by the telemetry data or video data.

For each medical practitioner of the set, the collaborative medical platform 140 determines 1306 a probability of a medical practitioner of the set being connected to the telemetry data or video data. In various embodiments, the collaborative medical platform determines 1306 the probability for a medical practitioner of the set based on the telemetry data or video data and characteristics of the medical practitioner of the set. For example, the collaborative medical platform 140 applies a trained practitioner prediction model to each medical practitioner of the set and the telemetry data or video data, with the practitioner prediction model determining 1306 the probability of the medical practitioner of the set being connected to the telemetry data or video data, as further described above in conjunction with FIG. 2. Based on the determined probabilities, the collaborative medical platform 140 selects 1308 a medical practitioner of the set. For example, the collaborative medical platform 140 selects 1308 a medical practitioner of the set having a maximum determined probability.

In various embodiments, the collaborative medical platform 140 stores 1310 a connection between the selected medical practitioner and the telemetry data or video data. For example, the collaborative medical platform 140 automatically stores 1310 the connection between the selected medical practitioner and the telemetry data or video data. Alternatively, the collaborative medical platform 140 transmits a prompt to a client device 150 of the selected medical practitioner including descriptive information of the telemetry data or video data including a request for the selected medical practitioner to confirm a connection to the telemetry data or video data. In response to receiving the confirmation from the selected medical practitioner, the collaborative medical platform 140 stores 1310 the connection between the selected medical practitioner and the telemetry data or video data.

The collaborative medical platform 140 may transmit a request for supplemental information for the telemetry data or video data to the client device 150 of the selected medical practitioner in response to storing 1310 the connection between the selected medical practitioner and the telemetry data or video data. For example, the collaborative medical platform 140 generates a request for supplemental information by applying a generative model, such as a large language model, to the telemetry data or video data and one or more characteristics of the selected medical practitioner (e.g., characteristics from the user profile of the selected medical practitioner). The generative model generates one or more requests that are subsequently presented to the selected medical practitioner. Alternatively, the collaborative medical platform 140 maintains one or more predefined requests and presents one or more of the predefined requests in response to storing 1310 the connection between the selected medical practitioner and the telemetry data or video data. The collaborative medical platform 140 stores received supplemental data in association with the selected medical practitioner and with the telemetry data or video data, as further described above in conjunction with FIG. 2.

By determining 1306 probabilities of each medical practitioner of the set being connected to the telemetry data or video data, the collaborative medical platform 140 leverages stored characteristics of medical practitioners to reduce an amount of information that the collaborative medical platform 140 analyzes to connect a medical practitioner with the telemetry data or video data. This allows the collaborative medical platform 140 to receive telemetry data or video data that does not include information identifying a medical practitioner or a medical procedure and to identify a medical practitioner connected to the telemetry data or video data. Identification of a medical practitioner based on probabilities of being connected to telemetry data or video data using characteristics of medical practitioners allows locations to comply with data privacy restrictions on information identifying patients when transmitting telemetry data or video data to the collaborative medical platform 140, while allowing the collaborative medical platform 140 to identify a medical practitioner to receive data the collaborative medical platform 140 generates based on the telemetry data or video data.

The described embodiments incorporate multiple technical improvements that improve the functioning of computer systems, machine learning techniques, data management systems (particularly as related to healthcare data management), computer-based user interfaces, robotic and/or other medical instrumentation systems, and other technologies and technical fields. For example, the disclosed embodiments improve data availability by using disparate types of data from various sources to predict information (such as an identity of a medical practitioner) that may otherwise be restricted from a medical system due to data privacy compliance.

The described embodiments furthermore include improvements in machine learning methods in that they combine information from disparate data sources including medical equipment telemetry data, video data, and mobile device data to improve predictive power relative to traditional machine learning techniques. Further still, by linking telemetry data and video associated with medical procedures to a medical practitioner, the described embodiments enable generation of various notifications, recommendations, or other content tailored to specific medical practitioners that enable them to improve their practice, resulting in better patient outcomes.

Furthermore, the described embodiments include technical improvements in the field of robotic-assisted surgery in that robotic systems learn connections between medical telemetry data and a medical practitioner, allowing the robotic systems to learn nuances of how a particular medical practitioner operates and interacts with such robotic systems, enabling such robotic systems to be configured in ways specifically tailored to the particular medical practitioner (e.g., by automatically controlling one or more settings of a surgical robot based on learned behaviors of a medical practitioner). This can in turn improve patient outcomes and represents technical improvements in the medical field.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

As used herein, unless expressly stated to the contrary, “or” refers to an inclusive “or” and not to an exclusive “or.” For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a non-limiting example, the condition “A, B, or C” is satisfied when A and B are true (or present) and C is false (or not present). Similarly, as another non-limiting example, the condition “A, B, or C” is satisfied when A is true (or present) and B and C are false (or not present).

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for connecting a medical practitioner associated with a medical procedure to telemetry data or video data received by an online collaborative medical platform, the method comprising:
receiving, at the collaborative medical platform, telemetry data or video data captured during performance of the medical procedure;
selecting a set of medical practitioners each having a user profile maintained by the collaborative medical platform based on the telemetry data or video data;
determining a probability of each medical practitioner of the set having a connection to the telemetry data or video data, the probability of a medical practitioner of the set being connected to the telemetry data or video data based on characteristics of the medical practitioner of the set from a user profile maintained by the collaborative medical platform and the telemetry data or video data;
selecting a medical practitioner of the set based on the determined probabilities; and
storing a connection between the selected medical practitioner and the telemetry data or video data at the collaborative medical platform.

2. The method of claim 1, wherein storing the connection between the selected medical practitioner and the telemetry data or video data at the collaborative medical platform comprises:
transmitting a prompt including descriptive information about the telemetry data or video data and a request to confirm the connection between the selected medical practitioner and the telemetry data or video data from the collaborative medical platform to a client device of the selected medical practitioner; and
storing the connection between the selected medical practitioner and the telemetry data or video data in response to receiving a confirmation from the selected medical practitioner in response to the prompt.

3. The method of claim 1, wherein determining the probability of each medical practitioner of the set having the connection to the telemetry data or video data comprises:
applying a practitioner prediction model to each medical practitioner of the set and the telemetry data or video data, the practitioner prediction model determining the probability of the medical practitioner of the set, and the practitioner prediction model trained by:
obtaining a training dataset including a plurality of training examples, each training example including training telemetry data or training telemetry data and characteristics of a training user and having a label indicating whether the training medical practitioner is connected to the training telemetry data or to the training video data;
applying the practitioner prediction model to each training example of the training dataset to generate a predicted probability of the training medical practitioner being connected to the training telemetry data or to the training video data;
scoring the practitioner prediction model using a loss function, the predicted probability of the training medical practitioner being connected to the training telemetry data or to the training video data and the label of the training example; and
updating one or more parameters of the practitioner prediction model by backpropagation based on the scoring until one or more criteria are satisfied.

4. The method of claim 1, wherein selecting the medical practitioner of the set based on the determined probabilities comprises:
selecting a medical practitioner of the set having a maximum determined probability.

5. The method of claim 1, wherein a characteristic of the medical practitioner of the set from the user profile maintained by the collaborative medical platform comprises an indication whether the medical practitioner of the set accessed the collaborative medical platform via a client device during a time interval corresponding to the telemetry data or video data.

6. The method of claim 1, wherein a characteristic of the medical practitioner of the set comprises a location associated with the medical practitioner and the telemetry data or video data includes a location where the telemetry data or video data was captured.

7. The method of claim 1, wherein selecting the set of medical practitioners each having a user profile maintained by the collaborative medical platform based on the telemetry data or video data comprises:
identifying a location from which the telemetry data or video data was received; and
selecting medical practitioners having the identified location included in their user profiles maintained by the collaborative medical platform.

8. The method of claim 1, further comprising:
transmitting one or more interfaces including one or more requests for supplemental information about the telemetry data or video data from the collaborative medical platform to a client device of the selected medical practitioner in response to storing the connection between the selected medical practitioner and the telemetry data or video data.

9. The method of claim 8, further comprising:
storing supplemental information received from the selected medical practitioner via the one or more interfaces in association with the telemetry data or video data and with the selected medical practitioner.

10. The method of claim 1, wherein the telemetry data or video data does not include information identifying one or more medical practitioners associated with the telemetry data or the video data or identifying a medical procedure during which the telemetry data or video data was captured.

11. A non-transitory computer readable storage medium having instructions encoded thereon for connecting a medical practitioner associated with a medical procedure to telemetry data or video data received by an online collaborative medical platform, the instructions, when executed by one or more processors, cause the one or more processors to perform steps comprising:
receiving, at the collaborative medical platform, telemetry data or video data captured during performance of the medical procedure;
selecting a set of medical practitioners each having a user profile maintained by the collaborative medical platform based on the telemetry data or video data;
determining a probability of each medical practitioner of the set having a connection to the telemetry data or video data, the probability of a medical practitioner of the set being connected to the telemetry data or video data based on characteristics of the medical practitioner of the set from a user profile maintained by the collaborative medical platform and the telemetry data or video data;
selecting a medical practitioner of the set based on the determined probabilities; and
storing a connection between the selected medical practitioner and the telemetry data or video data at the collaborative medical platform.

12. The non-transitory computer readable storage medium of claim 11, wherein storing the connection between the selected medical practitioner and the telemetry data or video data at the collaborative medical platform comprises:
transmitting a prompt including descriptive information about the telemetry data or video data and a request to confirm the connection between the selected medical practitioner and the telemetry data or video data from the collaborative medical platform to a client device of the selected medical practitioner; and
storing the connection between the selected medical practitioner and the telemetry data or video data in response to receiving a confirmation from the selected medical practitioner in response to the prompt.

13. The non-transitory computer readable storage medium of claim 11, wherein determining the probability of each medical practitioner of the set having the connection to the telemetry data or video data comprises:
applying a practitioner prediction model to each medical practitioner of the set and the telemetry data or video data, the practitioner prediction model determining the probability of the medical practitioner of the set, and the practitioner prediction model trained by:
obtaining a training dataset including a plurality of training examples, each training example including training telemetry data or training telemetry data and characteristics of a training user and having a label indicating whether the training medical practitioner is connected to the training telemetry data or to the training video data;
applying the practitioner prediction model to each training example of the training dataset to generate a predicted probability of the training medical practitioner being connected to the training telemetry data or to the training video data;
scoring the practitioner prediction model using a loss function, the predicted probability of the training medical practitioner being connected to the training telemetry data or to the training video data and the label of the training example; and
updating one or more parameters of the practitioner prediction model by backpropagation based on the scoring until one or more criteria are satisfied.

14. The non-transitory computer readable storage medium of claim 11, wherein selecting the medical practitioner of the set based on the determined probabilities comprises:
selecting a medical practitioner of the set having a maximum determined probability.

15. The non-transitory computer readable storage medium of claim 11, wherein a characteristic of the medical practitioner of the set from the user profile maintained by the collaborative medical platform comprises an indication whether the medical practitioner of the set accessed the collaborative medical platform via a client device during a time interval corresponding to the telemetry data or video data.

16. The non-transitory computer readable storage medium of claim 11, wherein a characteristic of the medical practitioner of the set comprises a location associated with the medical practitioner and the telemetry data or video data includes a location where the telemetry data or video data was captured.

17. The non-transitory computer readable storage medium of claim 11, wherein selecting the set of medical practitioners each having a user profile maintained by the collaborative medical platform based on the telemetry data or video data comprises:
identifying a location from which the telemetry data or video data was received; and
selecting medical practitioners having the identified location included in their user profiles maintained by the collaborative medical platform.

18. The non-transitory computer readable storage medium of claim 11, further having instructions encoded thereon that, when executed by the one or more processors, cause the one or more processors to perform steps comprising:
transmitting one or more interfaces including one or more requests for supplemental information about the telemetry data or video data from the collaborative medical platform to a client device of the selected medical practitioner in response to storing the connection between the selected medical practitioner and the telemetry data or video data.

19. The non-transitory computer readable storage medium of claim 18, further having instructions encoded thereon that, when executed by the one or more processors, cause the one or more processors to perform steps comprising:
storing supplemental information received from the selected medical practitioner via the one or more interfaces in association with the telemetry data or video data and with the selected medical practitioner.

20. The non-transitory computer readable storage medium of claim 11, wherein the telemetry data or video data does not include information identifying one or more medical practitioners associated with the telemetry data or the video data or identifying a medical procedure during which the telemetry data or video data was captured.
